(19) United States
(12) Patent Application Publication
(10) Pub. No.: US 2017/0158699 A1
(43) Pub. Date: Jun. 8, 2017

(54) ADDITION SALTS OF (S)-2-(1-(6-AMINO-5-CYANOPYRIMIDIN-4-YLAMINO)ETHYL)-4-OXO-3-PHENYL-3,4-DIHYDROPYRROLO[1,2-F][1,2,4]TRIAZINE-5-CARBONITRILE

(71) Applicant: Almirall, S.A., Barcelona (ES)

(72) Inventors: Francesc CARRERA CARRERA, Barcelona (ES); Juan Bautista PEREZ GARCIA, Barcelona (ES); Bernat VIDAL JUAN, Barcelona (ES); Francisco SANCHEZ IZQUIEROD, Barcelona (ES); Maria Carme SERRA COMA, Barcelona (ES)

(21) Appl. No.: 15/313,762

(22) PCT Filed: May 21, 2015
(86) PCT No.: PCT/EP2015/061307

§ 371 Date: Nov. 23, 2016

(30) Foreign Application Priority Data
May 27, 2014 (EP) 14382192.4
Oct. 17, 2014 (EP) 14382400.1
Oct. 17, 2014 (EP) 14382401.9

(51) Int. Cl. C07D 487/04; A61K 31/53; C07C 309/30; C07C 309/04; C07C 309/35

(52) U.S. Cl. CPC C07D 487/04; C07C 309/04; C07C 309/35; C07C 309/30; A61K 31/53; C07B 2200/13

(57) ABSTRACT

The present invention is directed to novel pharmaceutically acceptable, addition salts of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile with sulfonic acid derivatives, in particular with methanesulfonic acid, naphthalene-2-sulfonic acid and para-toluenesulfonic acid, and pharmaceutically acceptable solvates thereof, and their use as Phosphoinositide 3-Kinase (PI3K) inhibitors.

### FIELD OF THE INVENTION

The present invention is directed to novel crystalline, stable and pharmaceutically acceptable, addition salts of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile with sulfonic acid derivatives, in particular with methanesulfonic acid, naphthalene-2-sulfonic acid and para-toluenesulfonic acid, and pharmaceutically acceptable solvates thereof. The invention is also directed to pharmaceutical compositions comprising the salts, methods of using them to treat, prevent or suppress diseases and disorders susceptible to be ameliorated by inhibition of Phosphoinositide 3-Kinase (PI3K).

### BACKGROUND OF THE INVENTION

When cells are activated by extracellular stimuli, intracellular signalling cascades involving the regulation of second messengers are initiated that eventually produce a response of the cell to the stimuli. Phosphoinositide 3-Kinases (PI3Ks) are among the enzymes involved in early signalling events to a plethora of different types of stimuli. PI3Ks phosphorylate the 3-hydroxyl group of the inositol ring of phosphatidylinositol (Ptdlns), Ptdlns-4-phosphate (Ptdlns4P), and Ptdlns-4,5-bisphosphate (Ptdlns(4,5)P2). The resulting 3-phosphoinositides mediate correct localization and subsequent activation of a number of downstream effector proteins that bind to the lipids via specific lipid binding sequences such as the pleckstrin homology (PH) domain (Vanhaesebroeck B, 2010, Nat Rev Mol Cell Biol 5:11381-6).

The PI3K family is divided into 3 different classes (PI3K class I, class II, and class III), depending on substrate preference and structural features.

The best characterized is the PI3K class I with the preferential substrate Ptdlns-(4,5)P2. It englobes 4 different isoforms which originally were further subdivided into class IA (p110a, p110b, p110d), binding to a p85 type of regulatory subunit, and class IB (p110g) which is regulated by p101 and p87 subunits. Whereas p110a (PI3Ka or PI3Kα) and p110b (PI3Kb or PI3Kβ) isoforms are expressed ubiquitously, p110g (PI3Kg or PI3Kγ) and especially p110d (PI3Kd or PI3Kδ) have a more restricted expression pattern and seem to play a major role in leukocytes (Kok K, Trends Biochem Science 34:115-127, 2009).

Conditions in which targeting of the PI3K pathway or modulation of the PI3 Kinases, particularly PI3Kd or PI3Kd/g, are contemplated to be therapeutically useful for the treatment or prevention of diseases include: respiratory diseases (asthma, chronic obstructive pulmonary disease (COPD), cystic fibrosis, bronchiectasis, cough, idiopathic pulmonary fibrosis, sarcoidosis), allergic diseases (allergic rhinitis), inflammatory or autoimmune diseases (rheumatoid arthritis, multiple sclerosis, amyotrophic lateral sclerosis, Crohn's disease, ulcerative colitis, systemic lupus erythematosis, myastenia gravias, acute disseminated encephalomyelitis, idiopathic thromocytopenic purpura, Sjoegren's syndrome, autoimmune hemolytic anemia, type I diabetes, psoriasis, acrodermatitis, angiodermatitis, atopic dermatitis, contact dermatitis, eczema, acne, chronic urticaria, scleroderma, cutaneous vasculitis, cutaneous lupus erythematosus, dermatomyositis and blistering diseases including but not limited to pemphigus vulgaris, bullous pemphigoid and epidermolysis bullosa), cardiovascular diseases; viral infection; metabolism/endocrine function disorders; neurological disorders and pain (such as pain associated with rheumatoid arthritis or osteoarthritis, back pain, general inflammatory pain, inflammatory neuropathic pain, trigeminal neuralgia or central pain) as well as in bone marrow and organ transplant rejection; myelo-dysplastic syndrome; myeloproliferative disorders (such as polycythemia vera, essential thrombocythemia or mielofibrosis); cancer and hematologic malignancies, leukemia, lymphomas and solid tumors (such as pancreatic cancer; bladder cancer; colorectal cancer; breast cancer; prostate cancer; renal cancer; hepatocellular cancer; lung cancer; ovarian cancer; cervical cancer; gastric cancer; esophageal cancer; head and neck cancer; non-small cell lung cancer and small-cell lung cancer; melanoma; neuroendocrine cancers; central nervous system cancers; brain tumors; bone cancer; soft tissue sarcoma; chronic lymphocytic leukemia, B-cell acute lymphoblastic leukemia, T-cell acute lymphoblastic leukaemia, non-hodgkins lymphoma, B-cell lymphoma, acute myeloid leukaemia; cutaneous T cell lymphoma, premalignant and malignant skin conditions including but not limited to basal cell carcinoma (BCC), squamous cell carcinoma (SCC) or actinic keratosis (AK)).

In view of the numerous conditions that are contemplated to benefit by treatment involving modulation of the PI3K pathway or modulation of the PI3 Kinases it is immediately apparent that new compounds that modulate PI3K pathways and use of these compounds should provide substantial therapeutic benefits to a wide variety of patients. Thus, several PI3K inhibitors are in clinical trials for the treatment or prevention of some of the diseases or disorders indicated above. See for example alpelisib (previously known as BYL-719), buparlisib (previously known as BKM 120 or NVP-BKM120), duvelisib (previously known as IPI-145 or INK-1197), idelalisib (previously known as GS-1101 or CAL-101), rigosertib sodium (previously known as ON-1910Na), or 6-(2-((4-amino-3-(3-hydroxyphenyl)-1H-pyrazolo[3,4-d]pyrimidin-1-yl)methyl)-3-(2-chlorobenzyl)-4-oxo-3,4-dihydroquinazolin-5-yl)-N, N-bis(2-methoxyethyl)hex-5-ynamide (also known as RV-1729).

Many organic and inorganic compounds can exist in different solid forms. They can be in the amorphous, i.e., disordered, or in the crystalline, i.e. ordered, state. Amorphous forms consist of disordered arrangements of molecules that do not possess a distinguishable crystal lattice. On the contrary, crystalline forms have different arrangements and/or conformations of the molecules in the crystal lattice. The polymorphism of any element or compound is the ability to crystallize as more than one distinct crystal species (McCrone, W. C., Phys. Chem. Org. Solid State, 1965, 2, 725-767).

Polymorphic forms of a drug substance can have different chemical and physical properties including melting point, chemical reactivity, apparent solubility, dissolution rate, optical and mechanical properties, vapour pressure and density. These properties can have a direct effect on the ability to process and/or manufacture the drug substance and the drug product, as well as on drug product stability, dissolution and bioavailability. Thus, polymorphism can affect the quality, safety and efficacy of the drug product and is therefore of fundamental importance (Giron D. et al, J. Therm. Anal. Cal. 2004, 77:709-747)

Since an applicant for a marketing authorisation for a medicinal product should demonstrate that a drug product can be manufactured reliably using a validated process and that the drug product exhibits adequate stability, formulators in the pharmaceutical industry should pay close attention to polymorphism to avoid phase conversion of the drug substance when exposed to different manufacturing processes, such as drying, milling, micronization, etc.

WO 2012/146666 discloses pyrrolotriazinone derivatives as potent PI3Ks inhibitors. Although these compounds have shown adequate pharmacological activity, some of the compounds exemplified in this International Patent Application present a complex polymorphic landscape with numerous crystalline forms.

Accordingly, there is a need for PI3Ks inhibitors, which are physically and chemically stable, with relative high melting point and which do not exhibit polymorphism. This would allow the material to be further manipulated, e.g. by drying, milling or by micronization without significant decomposition, loss of crystallinity or exhibiting any change in polymorphism to prepare pharmaceutical compositions and formulations.

### SUMMARY OF THE INVENTION

It has now been found that addition salts of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile with sulfonic acid derivatives, in particular with methanesulfonic acid, naphthalene-2-sulfonic acid and para-toluenesulfonic acid, and pharmaceutically acceptable solvates thereof, are stable and can be obtained in a crystalline form which has a relatively high melting point and does not exhibit any change in polymorphism.

Thus, the present invention provides pharmaceutically acceptable crystalline addition salts of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile with sulfonic acid derivatives selected from methanesulfonic acid, naphthalene-2-sulfonic acid and para-toluenesulfonic acid, and pharmaceutically acceptable solvates thereof.

The invention also provides a pharmaceutical composition comprising a salt of the invention and a pharmaceutically acceptable carrier. The invention further provides pharmaceutical compositions as defined before and a therapeutically effective amount of one or more other therapeutic agents. The invention further provides combinations comprising a salt of the invention and a therapeutically effective amount of one or more other therapeutic agents.

The invention also provides a method of treatment of a pathological condition or disease susceptible to amelioration by inhibition of Phosphoinositide 3-Kinase (PI3K), in particular wherein the pathological condition or disease is selected from respiratory diseases; allergic diseases; inflammatory or autoimmune-mediated; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors; more in particular wherein the pathological condition or disease is selected from leukemia, lymphomas and solid tumors, rheumatoid arthritis, multiple sclerosis, amyotrophic lateral sclerosis, Crohn's disease, ulcerative colitis, systemic lupus erythematosis, autoimmune hemolytic anemia, type I diabetes, cutaneous vasculitis, cutaneous lupus erythematosus, dermatomyositis, blistering diseases including but not limited to pemphigus vulgaris, bullous pemphigoid and epidermolysis bullosa, asthma, chronic obstructive pulmonary disease (COPD), cystic fibrosis, bronchiectasis, cough, idiopathic pulmonary fibrosis, sarcoidosis, allergic rhinitis, atopic dermatitis, contact dermatitis, eczema, psoriasis, basal cell carcinoma, squamous cell carcinoma and actinic keratosis; comprising administering a therapeutically effective amount of a salt of the invention.

The invention also provides a method of treatment of a pathological condition or disease susceptible to amelioration by inhibition of Phosphoinositide 3-Kinase (PI3K), in particular wherein the pathological condition or disease is as defined before, comprising administering a therapeutically effective amount of a pharmaceutical composition comprising a salt of the invention and a pharmaceutically-acceptable carrier, a pharmaceutical composition comprising a salt of the invention, a pharmaceutically-acceptable carrier and a therapeutically effective amount of one or more other therapeutic agents as defined before.

The invention also provides a method of treatment of a pathological condition or disease susceptible to amelioration by inhibition of Phosphoinositide 3-Kinase (PI3K), in particular wherein the pathological condition or disease is as defined before, comprising administering a therapeutically effective amount of a combination comprising a salt of the invention and one or more other therapeutic agents.

The invention also provides a salt of the invention as described herein, a pharmaceutical composition comprising a salt of the invention and a pharmaceutically acceptable carrier, a pharmaceutical composition as defined above together with a therapeutically effective amount of one or more other therapeutic agents or combination of a salt of the invention together with a therapeutically effective amount of one or more other therapeutic agents, for use in the treatment of a pathological condition or disease susceptible to amelioration by inhibition of Phosphoinositide 3-Kinase (PI3K); in particular wherein the pathological condition or disease from respiratory diseases; allergic diseases; inflammatory or autoimmune-mediated; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors; more in particular wherein the pathological condition or disease is selected from leukemia, lymphomas and solid tumors, rheumatoid arthritis, multiple sclerosis, amyotrophic lateral sclerosis, Crohn's disease, ulcerative colitis, systemic lupus erythematosis, autoimmune hemolytic anemia, type I diabetes, cutaneous vasculitis, cutaneous lupus erythematosus, dermatomyositis, blistering diseases including but not limited to pemphigus vulgaris, bullous pemphigoid and epidermolysis bullosa, asthma, chronic obstructive pulmonary disease (COPD), cystic fibrosis, bronchiectasis, cough, idiopathic pulmonary fibrosis, sarcoidosis, allergic rhinitis, atopic dermatitis, contact dermatitis, eczema, psoriasis, basal cell carcinoma, squamous cell carcinoma and actinic keratosis.

The invention also provides the use of the salt of the invention, a pharmaceutical composition comprising a salt of the invention and a pharmaceutically acceptable carrier, a pharmaceutical composition as defined above together with a therapeutically effective amount of one or more other therapeutic agents or a combination of a salt of the invention together with one or more other therapeutic agents, for the manufacture of a formulation or medicament for treating these diseases.

### BRIEF DESCRIPTION OF THE FIGURES

FIG. 1 illustrates the X-Ray Powder Diffraction (XRPD) diffractogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate.

FIG. 2 illustrates the Differential Scanning Calorimetry (DSC) thermogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate.

FIG. 3 illustrates the Gravimetric Vapour Sorption (GVS) isotherm of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate.

FIG. 4 illustrates the Proton Nuclear Magnetic Resonance (¹H NMR) spectrum of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate.

FIG. 5 illustrates the X-Ray Powder Diffraction (XRPD) diffractogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate.

FIG. 6 illustrates the Differential Scanning Calorimetry (DSC) thermogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate.

FIG. 7 illustrates the Gravimetric Vapour Sorption (GVS) isotherm of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate.

FIG. 8 illustrates the Proton Nuclear Magnetic Resonance (¹H NMR) spectrum of (S)-2-(1-(6-amino-5-cyanopyrimidin-4-ylamino)ethyl)-4-oxo-3-phenyl-3,4-di hydropyrrolo[1,2-f][1,2,4]triazine-5-carbonitrile naphthalene-2-sulfonate.

FIG. 9 illustrates the X-Ray Powder Diffraction (XRPD) diffractogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate.

FIG. 10 illustrates the Differential Scanning Calorimetry (DSC) thermogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate.

FIG. 11 illustrates the Gravimetric Vapour Sorption (GVS) isotherm of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate.

FIG. 12 illustrates the Proton Nuclear Magnetic Resonance (¹H NMR) spectrum of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate.

FIG. 13 illustrates the X-Ray Powder Diffraction (XRPD) diffractogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate monohydrate.

FIG. 14 illustrates the Thermo-Gravimetric analysis (TGA) and Differential Scanning Calorimetry (DSC) thermograms of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate monohydrate.

FIG. 15 illustrates the Proton Nuclear Magnetic Resonance (¹H NMR) spectrum of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate monohydrate.

### DETAILED DESCRIPTION OF THE INVENTION

When describing the salts, compositions, combinations and methods of the invention, the following terms have the following meanings, unless otherwise indicated.

The term “therapeutically effective amount” refers to an amount sufficient to effect treatment when administered to a patient in need of treatment.

The term “treatment” as used herein refers to the treatment of a disease or medical condition in a human patient which includes:

(a) preventing the disease or medical condition from occurring, i.e., prophylactic treatment of a patient;
(b) ameliorating the disease or medical condition, i.e., causing regression of the disease or medical condition in a patient;
(c) suppressing the disease or medical condition, i.e., slowing the development of the disease or medical condition in a patient; or
(d) alleviating the symptoms of the disease or medical condition in a patient.

The term “solvate” refers to a complex or aggregate formed by one or more molecules of a solute, i.e. a salt of the invention or a pharmaceutically-acceptable salt thereof, and one or more molecules of a solvent. Such solvates are typically crystalline solids having a substantially fixed molar ratio of solute and solvent. Representative solvents include by way of example, water, ethanol, isopropanol and the like. When the solvent is water, the solvate formed is a hydrate.

The term “pharmaceutically (or physiologically) acceptable carrier (or diluent)” refers to a carrier or diluent that does not cause significant irritation to an organism and does not abrogate the biological activity and properties of the administered compound.

(S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile, which has the structure of formula (I), as well as a process for its manufacture, is described in the International Patent Application No. WO 2012/146666.

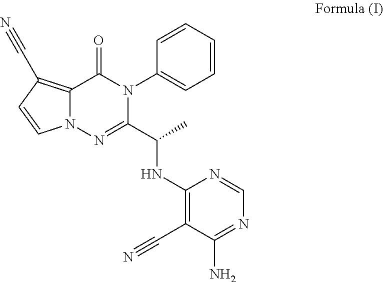 [SMILES: C[C@H](Nc1ncnc(N)c1C#N)c1nn2ccc(C#N)c2c(=O)n1-c1ccccc1]

One embodiment of the present invention refers to a pharmaceutically acceptable crystalline addition salt of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile with sulfonic acid derivatives selected from methanesulfonic acid, naphthalene-2-sulfonic acid and para-toluenesulfonic acid, and pharmaceutically acceptable solvates thereof.

In a particular embodiment of the present invention the addition salt is (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate, and pharmaceutically acceptable solvates thereof.

Typically, methanesulfonic acid (CAS RN 75-75-2) is a colourless liquid with the molecular formula CH₄O₃S (molecular weight of 96.11 g/mol). Salts of methanesulfonic acid are known as methanesulfonates, mesilates (International Nonproprietary Name or INN) or mesylates (United States Adopted Name or USAN).

In another particular embodiment of the present invention the addition salt is (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate, and pharmaceutically acceptable solvates thereof.

Typically, naphthalene-2-sulfonic acid (CAS RN 120-18-3) is a solid at 20° C. with the molecular formula C₁₀H₈O₃S (molecular weight of 208.24 g/mol). Salts of naphthalene-2-sulfonic acid are known as naphthalene-2-sulfonates, napsilates (INN) or napsylates (USAN).

In still another particular embodiment of the present invention the addition salt is (S)-2-(1-(6-amino-5-cyanopyrimidin-4-ylamino)ethyl)-4-oxo-3-phenyl-3,4-di hydropyrrolo[1,2-f][1,2,4]triazine-5-carbonitrile para-toluenesulfonate, and pharmaceutically acceptable solvates thereof.

Typically, para-toluenesulfonic acid (CAS RN 104-15-4) or tosylic acid is a solid at 20° C. with the molecular formula C₇H₈O₃S (molecular weight of 172.20 g/mol). Salts of para-toluenesulfonic acid are known as para-toluenesulfonates, tosilates (INN) or tosylates (USAN).

In still another particular embodiment of the present invention the addition salt is (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile, para-toluenesulfonate monohydrate.

In a particular preferred embodiment of the present invention the addition salt is (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate, and pharmaceutically acceptable solvates thereof.

The invention also encompasses pharmaceutical compositions comprising a therapeutically effective amount of a salt as hereinabove defined and a pharmaceutically acceptable carrier.

In an embodiment of the present invention the pharmaceutical composition further comprises a therapeutically effective amount of one or more other therapeutic agents.

The invention is also directed to combinations comprising a salt of the invention and a therapeutically effective amount of one or more other therapeutic agents. The invention is also directed to pharmaceutical compositions comprising such combinations.

The invention is also directed to a salt of the invention as described herein, a pharmaceutical composition comprising a salt as hereinabove defined and a pharmaceutically acceptable carrier, a pharmaceutical composition as hereinabove defined together with a therapeutically effective amount of one or more other therapeutic agents, or a combination of a salt of the invention together with a therapeutically effective amount of one or more other therapeutic agents for use in the treatment of a pathological condition or disease susceptible to amelioration by inhibition of Phosphoinositide 3-Kinase (PI3K); in particular wherein the pathological condition or disease is selected from respiratory diseases; allergic diseases; inflammatory or autoimmune-mediated; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors; more in particular wherein the pathological condition or disease is selected from leukemia, lymphomas and solid tumors, rheumatoid arthritis, multiple sclerosis, amyotrophic lateral sclerosis, Crohn's disease, ulcerative colitis, systemic lupus erythematosis, autoimmune hemolytic anemia, type I diabetes, cutaneous vasculitis, cutaneous lupus erythematosus, dermatomyositis, blistering diseases including but not limited to pemphigus vulgaris, bullous pemphigoid and epidermolysis bullosa, asthma, chronic obstructive pulmonary disease (COPD), cystic fibrosis, bronchiectasis, cough, idiopathic pulmonary fibrosis, sarcoidosis, allergic rhinitis, atopic dermatitis, contact dermatitis, eczema, psoriasis, basal cell carcinoma, squamous cell carcinoma and actinic keratosis.

The invention also encompasses the use of a salt of the invention as described herein, a pharmaceutical composition comprising a salt as hereinabove defined and a pharmaceutically acceptable carrier, a pharmaceutical composition as hereinabove defined together with a therapeutically effective amount of one or more other therapeutic agents, or a combination of a salt of the invention together with a therapeutically effective amount of one or more other therapeutic agents for the manufacture of a formulation or medicament for treating these diseases.

The invention also encompasses a method of treatment of a pathological condition or disease susceptible to amelioration by inhibition of Phosphoinositide 3-Kinase (PI3K), in particular wherein the pathological condition or disease is selected from respiratory diseases; allergic diseases; inflammatory or autoimmune-mediated; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors; more in particular wherein the pathological condition or disease is selected from leukemia, lymphomas and solid tumors, rheumatoid arthritis, multiple sclerosis, amyotrophic lateral sclerosis, Crohn's disease, ulcerative colitis, systemic lupus erythematosis, autoimmune hemolytic anemia, type I diabetes, cutaneous vasculitis, cutaneous lupus erythematosus, dermatomyositis, blistering diseases including but not limited to pemphigus vulgaris, bullous pemphigoid and epidermolysis bullosa, asthma, chronic obstructive pulmonary disease (COPD), cystic fibrosis, bronchiectasis, cough, idiopathic pulmonary fibrosis, sarcoidosis, allergic rhinitis, atopic dermatitis, contact dermatitis, eczema, psoriasis, basal cell carcinoma, squamous cell carcinoma and actinic keratosis; comprising administering a therapeutically effective amount of a salt of the invention.

The invention also encompasses a method of treatment of these pathological conditions or diseases comprising administering a pharmaceutical composition comprising a salt as hereinabove defined and a pharmaceutically acceptable carrier, a pharmaceutical composition as hereinabove defined together with a therapeutically effective amount of one or more other therapeutic agents, or a combination of a salt of the invention together with a therapeutically effective amount of one or more other therapeutic agents.

General Synthetic Procedures

The salts of the invention can be prepared using the methods and procedures described herein, or using similar methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Processes for preparing salts of the invention are provided as further embodiments of the invention and are illustrated by the procedures below.

The salt of the invention can be synthesized from (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile and from methanesulfonic acid, naphthalene-2-sulfonic acid and para-toluenesulfonic acid, which are commercially available from, for example, Scharlau or Sigma-Aldrich.

Suitable inert diluents for this reaction include, but are not limited to, acetone, acetonitrile and tetrahydrofuran, and mixtures thereof, optionally containing water.

Upon completion of any of the foregoing reactions, the salt can be isolated from the reaction mixture by any conventional means such as precipitation, concentration, centrifugation and the like.

It will be appreciated that while specific process conditions (i.e. reaction temperatures, times, mole ratios of reactants, solvents, pressures, etc.) are given, other process conditions can also be used unless otherwise stated.

A salt of the invention typically contains between about 0.60 and 1.20 molar equivalents of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile per molar equivalent of the free base, more typically 0.85 and 1.15 molar equivalents of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile per molar equivalent of the free base, even more typically about 1 molar equivalent of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile per molar equivalent of the free base.

The molar ratios described in the methods of the invention can be readily determined by various methods available to those skilled in the art. For example, such molar ratios can be readily determined by ¹H NMR. Alternatively, elemental analysis and HPLC methods can be used to determine the molar ratio.

### EXAMPLES

General.

Reagents, starting materials, and solvents were purchased from commercial suppliers and used as received.

Crystallization tests of salts of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile with a broad range of pharmaceutically acceptable acids (hydrochloric acid, hydrobromic acid, phosphoric acid, sulphuric acid, L-aspartic acid, maleic acid, oxalic acid, benzene sulfonic acid, 1,2-ethane disulfonic acid, methanesulfonic acid, naphthalene-2-sulfonic acid, 1,5-naphthalene disulfonic acid and para-toluenesulfonic acid) in a range of different pharmaceutically acceptable solvents (acetone, acetonitrile and tetrahydrofuran) have been undertaken.

¹H NMR spectra of the solid obtained with L-aspartic acid indicated that a salt had not been formed.

The salts from hydrochloric acid, hydrobromic acid, phosphoric acid, sulphuric acid, maleic acid, oxalic acid, benzene sulfonic acid, 1,2-ethane disulfonic acid, and 1,5-naphthalene disulfonic acid rendered gels, amorphous solids, semicrystalline or crystalline solids. However, for the crystalline solids more than one X-ray powder diffraction (XRPD) pattern was observed for the same counterion, suggesting than these salts have multiple polymorphs.

Only the salts of the invention (methanesulfonic acid, naphthalene-2-sulfonic acid and para-toluenesulfonic acid) displayed a good thermal behaviour, had a relatively high melting point and showed an appropriate XRPD pattern before and after GVS determination (no change in form or crystallinity).

Particularly good methods to prepare the addition salts of the invention are illustrated in the following examples.

X-Ray Powder Diffraction (XRPD) patterns were collected on a Bruker D8 diffractometer using Cu Ka radiation (40 kV, 40 mA), θ-2θ goniometer, and divergence of V4 and receiving slits, a Ge monochromator and a Lynxeye detector. The instrument is performance checked using a certified Corundum standard (NIST 1976). The software used for data collection was Diffrac Plus XRD Commander v2.6.1 and the data were analysed and presented using Diffrac Plus EVA v13.0.0.2 or v15.0.0.0.

Samples were run under ambient conditions as flat plate specimens using powder as received. The sample was gently packed into a cavity cut into polished, zero-background (510) silicon wafer. The sample was rotated in its own plane during analysis. The details of the data collection were:
Angular range: 2 to 42° 2θ
Step size: 0.05° 2θ
Collection time: 0.5 s/step

The differential scanning calorimetry (DSC) thermograms were obtained using a TA Instruments Q2000 equipped with a 50 position auto-sampler. The calibration for thermal capacity was carried out using sapphire and the calibration for energy and temperature was carried out using certified indium. Typically 0.5-3 mg of each sample, in a pin-holed aluminium pan, was heated at 10° C./min from 25° C. to 300° C. (some runs up to 400° C.). A purge of dry nitrogen at 50 ml/min was maintained over the sample.

Proton Nuclear Magnetic Resonance (¹H NMR) spectra were collected on a Bruker 400 MHz instrument equipped with an auto-sampler and controlled by a DRX400 console. Automated experiments were acquired using ICON-NMR v4.0.7 running with Topspin v1.3 using the standard Bruker loaded experiments.

Thermo-Gravimetric analysis (TGA) isotherms were collected on a TA Instruments Q500 TGA, equipped with a 16 position autosampler. The instrument was temperature calibrated using certified Alumel and Nickel. Typically 3-10 mg of each sample was loaded onto a pre-tared aluminium DSC pan and heated at 10° C./min from ambient temperature to 350° C. A nitrogen purge at 60 ml/min was maintained over the sample.

Gravimetric Vapour Sorption (GVS; also known as Dynamic Vapour Sorption or DVS) isotherms were obtained using a SMS DVS Intrinsic moisture sorption analyser, controlled by DVS Intrinsic Control software v1.0.1.2. The sample temperature was maintained at 25° C. by the instrument controls. The humidity was controlled by mixing streams of dry and wet nitrogen, with a total flow rate of 200 mL/min The relative humidity was measured by a calibrated Rotronic probe (dynamic range of 1.0-100% RH), located near the sample. The weight change, (mass relaxation) of the sample as a function of % RH was constantly monitored by the microbalance (accuracy ±0.005 mg).

Typically 5-20 mg of sample were placed in a tared mesh stainless steel basket under ambient conditions. The sample was loaded and unloaded at 40% RH and 25° C. (typical room conditions). A moisture sorption isotherm was performed as outlined below (4 scans giving 2 complete cycles). The standard isotherm was performed at 25° C. at 10% RH intervals over a 0-90% RH range.

### Example 1: Preparation of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate

450 mg of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile were dissolved in 18 mL acetonitrile at 50° C. 1 equivalent methane sulfonic acid (methane sulfonic acid dissolved in tetrahydrofuran, 1M) was then added as a neat liquid. The sample was stirred (500 rpm) at 50° C. for 10 minutes. The sample was then cooled to 5° C. at 0.1° C. and held at 5° C. overnight until it was filtered. The sample was filtered using a PTFE autocup and then dried in a vacuum oven at 40° C. for 3 days.

The ¹H NMR spectra of the sample obtained confirmed the 1:1 stoichiometry of the solid with no residual solvents.

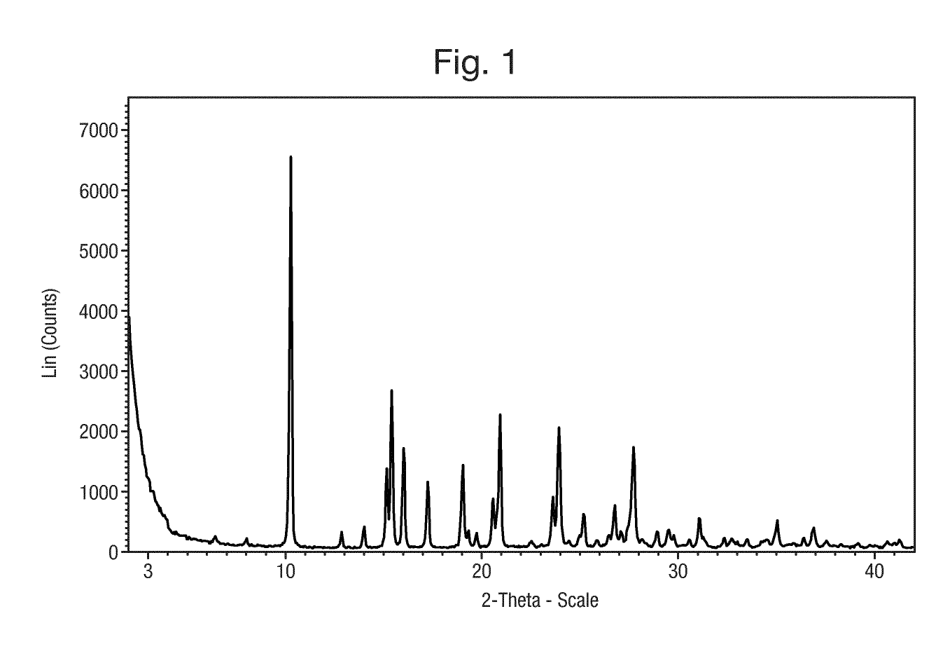

FIG. 1 illustrates the XRPD diffractogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate. The sample exhibits a good crystallinity.

The summary of the XRPD angles and relative intensities are given in Table 1 below.

TABLE 1

| Diffraction Angle (2-Theta °) | Relative Intensity (%) |
|---|---|
| 6.4 | 3.8 |
| 8.0 | 3.3 |
| 10.2 | 100.0 |
| 12.8 | 4.9 |
| 14.0 | 6.2 |
| 15.1 | 20.9 |
| 15.4 | 40.8 |
| 16.0 | 26.0 |
| 17.3 | 17.6 |
| 19.0 | 21.8 |
| 19.3 | 5.2 |
| 19.7 | 4.6 |
| 20.5 | 13.3 |
| 20.9 | 34.6 |
| 23.6 | 13.7 |
| 23.9 | 31.3 |
| 24.9 | 4.0 |

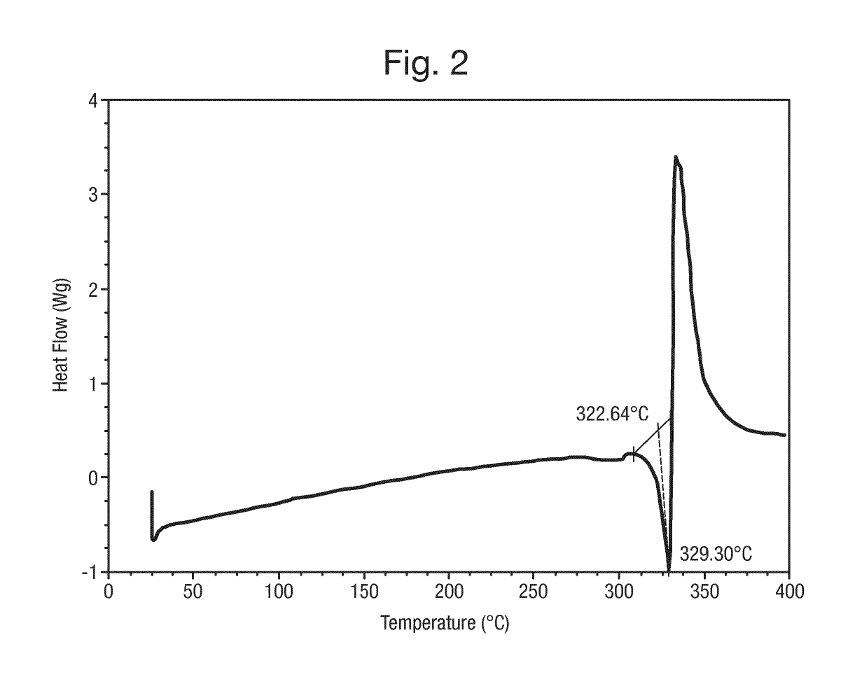

FIG. 2 illustrates the DSC thermogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate. The sample exhibits a characteristic high endotherm at onset 323° C. followed immediately by exotherm. This suggests that the sample melts/decomposes all at the same temperature and confirming the high stability of the sample until more than 300° C.

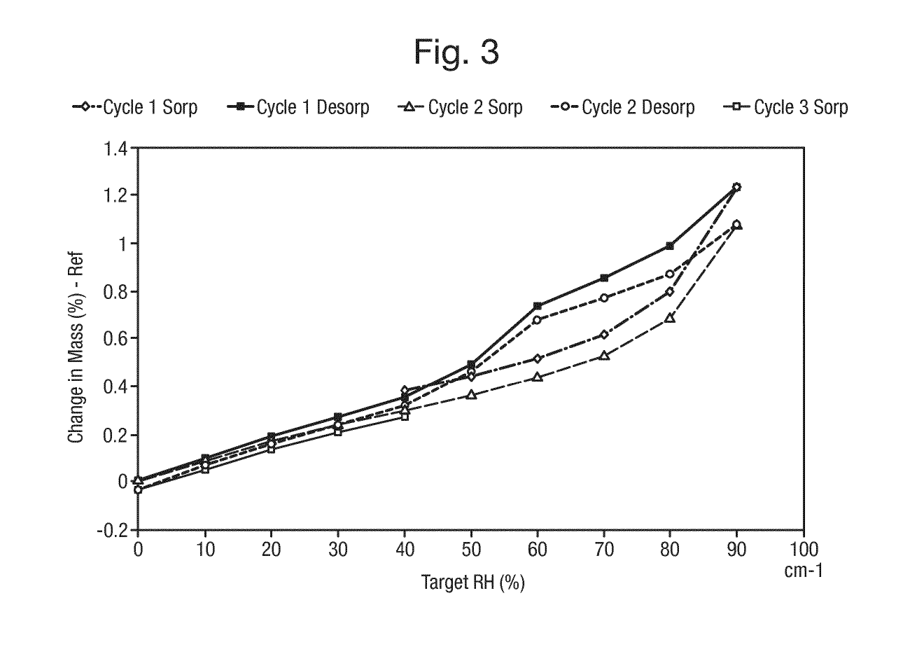

FIG. 3 illustrates the GVS isotherm of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate. Mass change was approx. 1.2% from 0-90% RH. This shows that the salt is not hygroscopic.

The sample showed no change in form or crystallinity (XRPD) after GVS measurement.

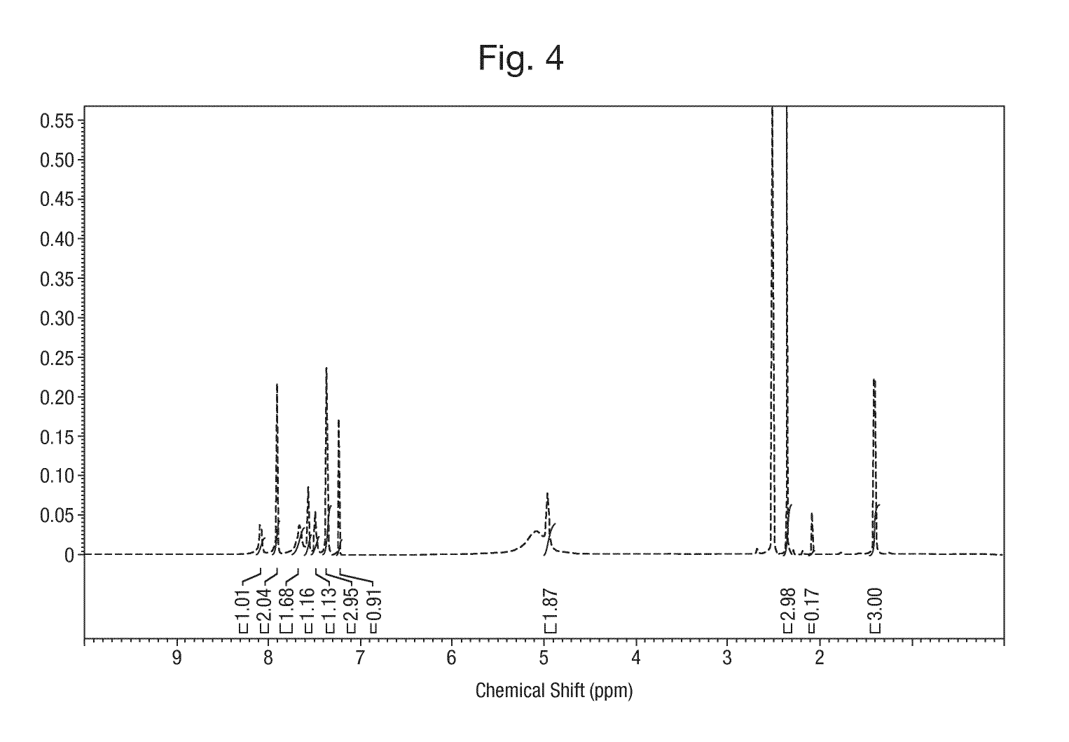

FIG. 4 corresponds to the ¹H-NMR spectrum of the methanesulfonate salt. It clearly shows a stoichiometry ratio of 1:1 free base/methanesulfonic acid, as inferred from the comparison between the integral values of the protons corresponding to the counterion and the free base.

### Example 2: Preparation of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate

320 mg of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile was dissolved in 9.6 mL) acetone at 50° C. 1 equivalent naphthalene-2-sulfonic acid was added as a 1M stock solution in ethanol. The sample was stirred (500 rpm) at 50° C. for 10 minutes. The sample was then cooled to 5° C. at 0.1° C. and held at 5° C. overnight until it was filtered. The sample was filtered using a PTFE autocup and then dried in a vacuum oven at 40° C. for 3 days before analysis by XRPD.

The ¹H NMR spectra of the sample obtained confirmed the 1:1 stoichiometry of the solid with no residual solvents.

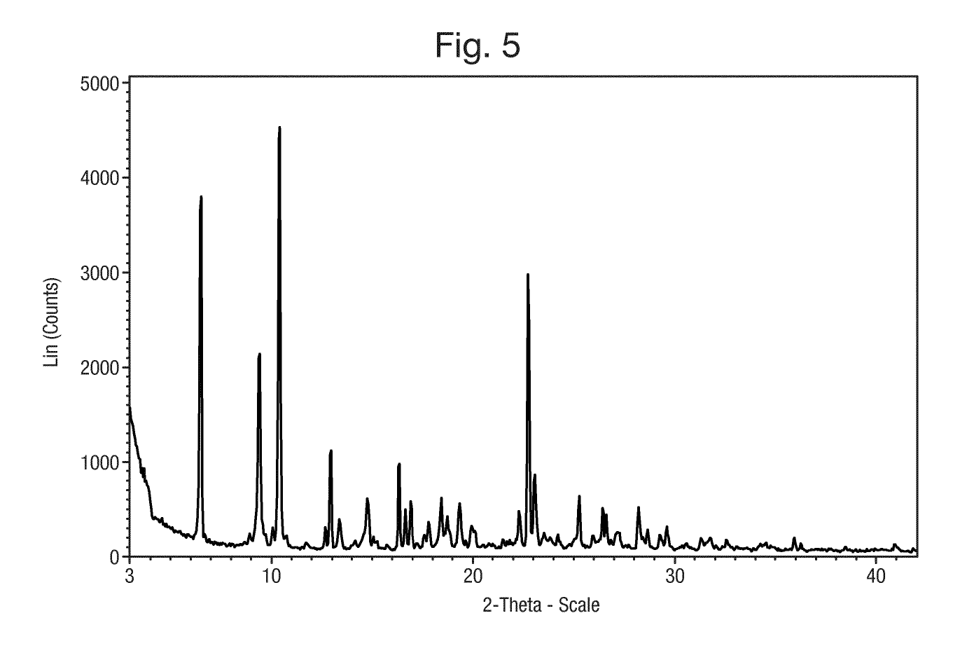

FIG. 5 illustrates the XRPD diffractogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate. The sample exhibits a good crystallinity.

The summary of the XRPD angles and relative intensities are given in Table 2 below.

TABLE 2

| Diffraction Angle (2-Theta °) | Relative Intensity (%) |
|---|---|
| 6.5 | 83.9 |
| 8.9 | 5.1 |
| 9.4 | 47.2 |
| 10.1 | 6.5 |
| 10.4 | 100.0 |
| 10.7 | 4.7 |
| 12.7 | 6.7 |
| 13.0 | 24.5 |
| 13.4 | 8.5 |
| 14.1 | 3.5 |
| 14.8 | 13.3 |
| 15.0 | 5.0 |
| 15.2 | 4.5 |
| 15.7 | 3.5 |
| 16.4 | 21.4 |
| 16.6 | 10.8 |
| 16.9 | 12.7 |
| 17.2 | 3.2 |
| 17.6 | 4.8 |
| 17.8 | 7.9 |
| 18.4 | 13.5 |
| 18.7 | 9.2 |
| 19.3 | 12.2 |
| 19.6 | 4.8 |
| 19.9 | 7.7 |
| 20.1 | 7.7 |
| 21.5 | 3.8 |
| 22.3 | 10.4 |
| 22.8 | 65.7 |
| 23.1 | 18.9 |
| 23.5 | 5.3 |
| 23.9 | 4.3 |
| 24.2 | 4.9 |

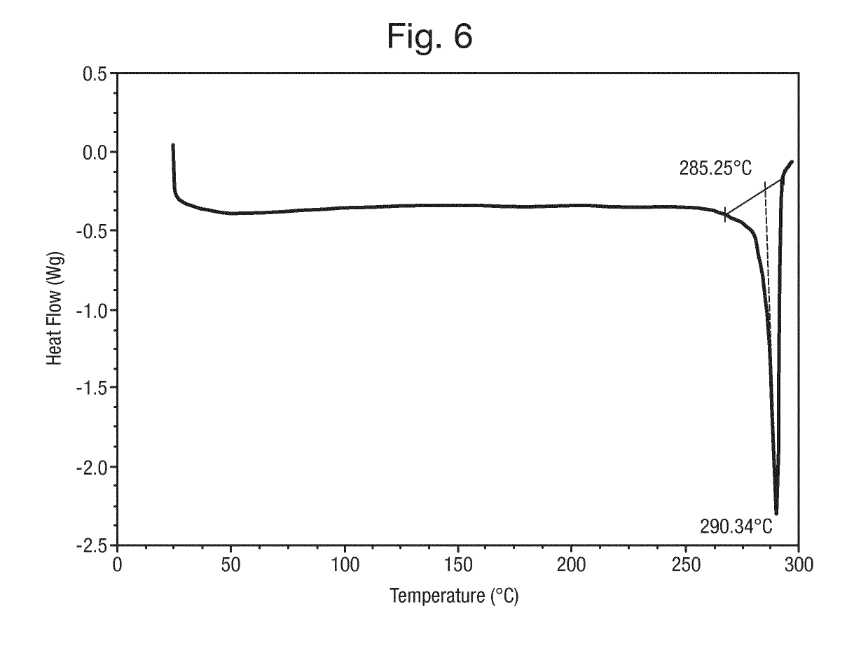

FIG. 6 illustrates the DSC thermogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate. The sample exhibits a characteristic high endotherm at onset 285° C. This confirms the high stability of the sample until more than 250° C.

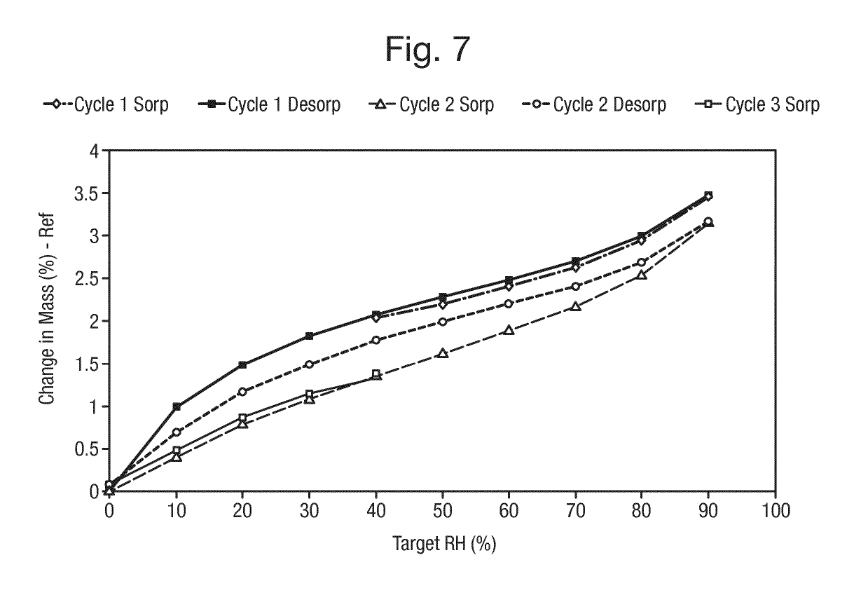

FIG. 7 illustrates the GVS isotherm of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate. Mass change was approx. 3.3% from 0-90% RH. This water sorption was reversible and no hydrates were formed during the GVS process.

The sample showed no change in form or crystallinity (XRPD) after GVS measurement.

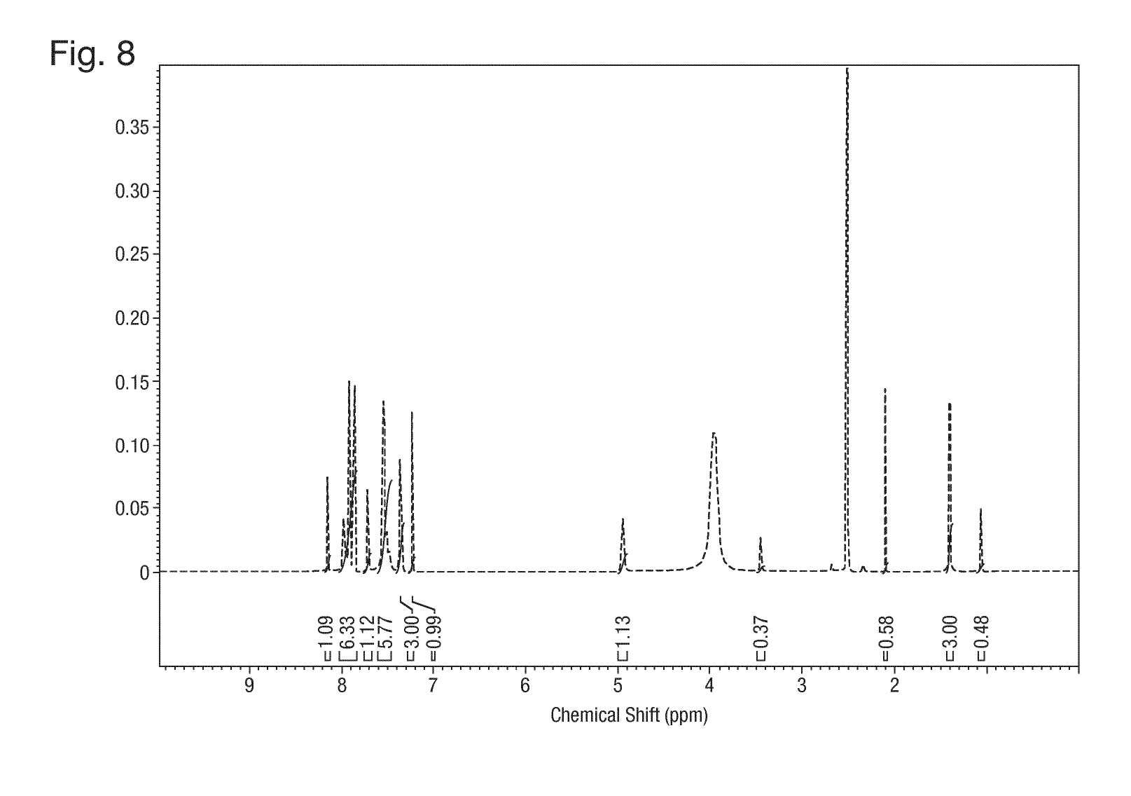

FIG. 8 corresponds to the ¹H-NMR spectrum of the naphthalene-2-sulfonate salt. It clearly shows a stoichiometry ratio of 1:1 free base/naphthalene-2-sulfonic acid, as inferred from the comparison between the integral values of the protons corresponding to the counterion and the free base.

### Example 3: Preparation of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate

450 mg (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile were dissolved in 18 mL acetonitrile at 50° C. 1 equivalent para-toluenesulfonic acid was then added as a 1M stock solution in tetrahydrofuran. The sample was stirred (500 rpm) at 50° C. for 10 minutes. The sample was then cooled to 5° C. at 0.1° C. and held at 5° C. overnight until it was filtered. The sample was filtered using a PTFE autocup and dried in a vacuum oven at 40° C. for 3 days. The sample was re-slurried in fresh acetonitrile at 50° C. for 1 hour before being filtered and dried in a vacuum oven at 40° C. overnight before analysis by XRPD.

The ¹H NMR spectra of the sample obtained confirmed the 1:1 stoichiometry of the solid with no residual solvents.

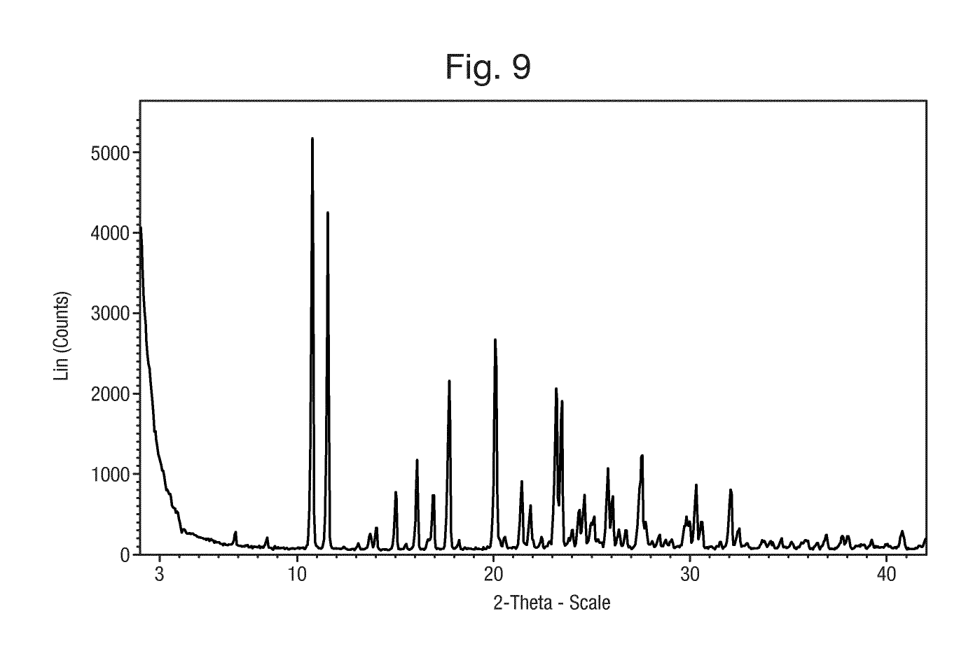

FIG. 9 illustrates the XRPD diffractogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate. The sample exhibits a good crystallinity.

The summary of the XRPD angles and relative intensities are given in Table 3 below.

TABLE 3

| Diffraction Angle (2-Theta °) | Relative Intensity (%) |
|---|---|
| 6.8 | 5.1 |
| 8.5 | 3.8 |
| 10.7 | 100.0 |
| 11.5 | 82.2 |
| 13.1 | 3.0 |
| 13.7 | 4.7 |
| 14.0 | 6.2 |
| 15.0 | 14.8 |
| 15.5 | 3.2 |
| 16.1 | 22.5 |
| 16.6 | 3.4 |
| 16.9 | 14.0 |
| 17.7 | 41.6 |
| 18.2 | 3.2 |
| 20.1 | 51.6 |
| 20.6 | 5.2 |
| 21.4 | 17.4 |
| 21.8 | 11.5 |
| 22.4 | 4.0 |
| 22.8 | 3.7 |
| 23.2 | 39.7 |
| 23.5 | 36.8 |
| 23.8 | 4.8 |
| 24.0 | 5.7 |
| 24.4 | 10.4 |
| 24.6 | 14.9 |

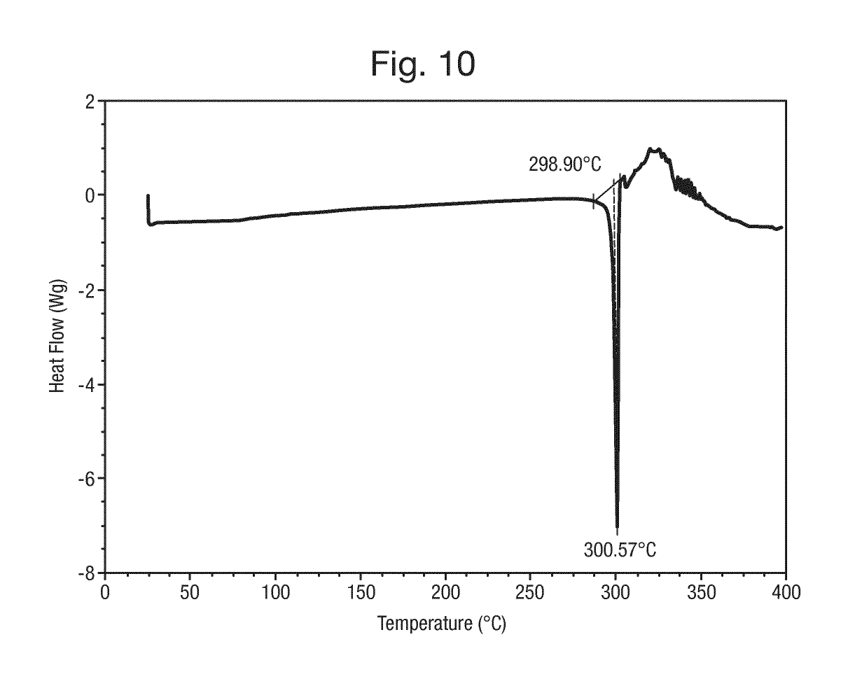

FIG. 10 illustrates the DSC thermogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate. The sample exhibits a characteristic high endotherm at onset 299° C. This confirms the high stability of the sample until more than 250° C.

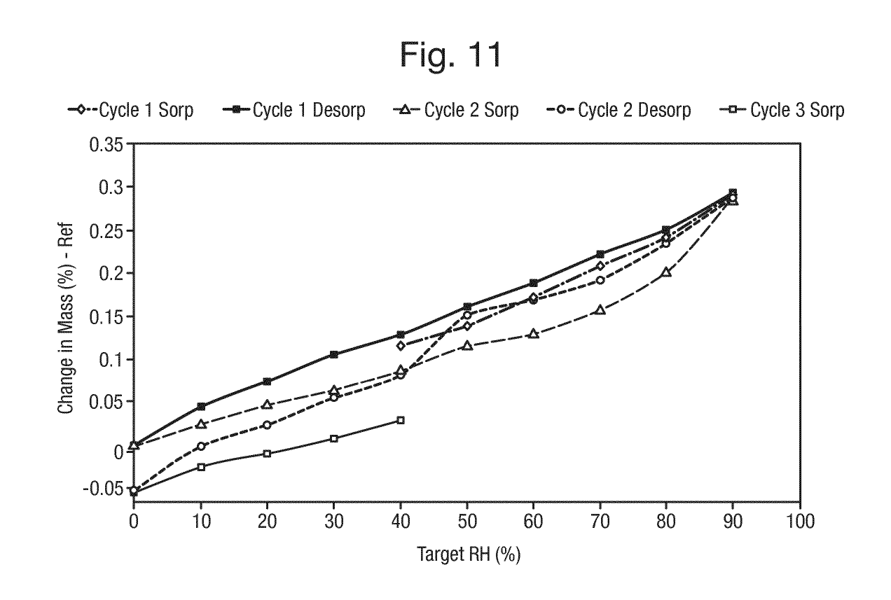

FIG. 11 illustrates the GVS isotherm of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate. Mass change was approx. 0.3% from 0-90% RH. This shows that the salt is not hygroscopic.

The sample showed no change in form or crystallinity (XRPD) after GVS measurement.

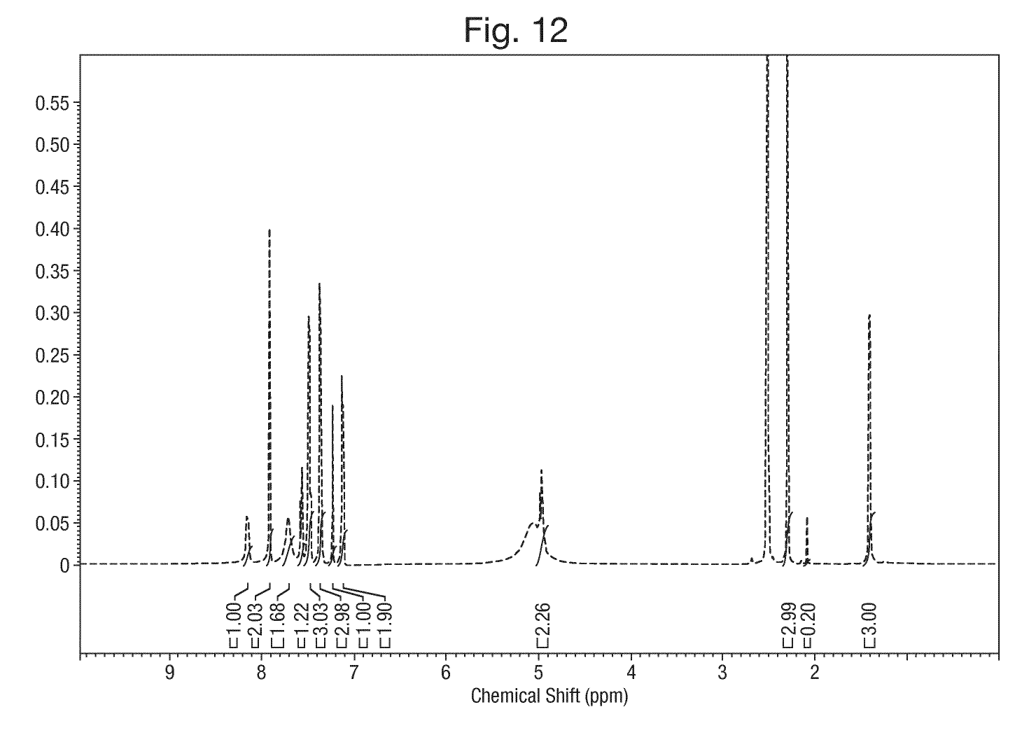

FIG. 12 corresponds to the ¹H-NMR spectrum of the para-toluenesulfonate salt. It clearly shows a stoichiometry ratio of 1:1 free base/para-toluenesulfonic acid, as inferred from the comparison between the integral values of the protons corresponding to the counterion and the free base.

### Example 4: Preparation of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate monohydrate

4a. The liquor from the second slurry of Example 3 was allowed to evaporate under ambient conditions and finally dried in a vacuum oven at 40° C. overnight before analysis by XRPD.

4b. 50 mg (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile were dissolved in 2 mL a solvent mixture (acetonitrile/10% water) at 50° C. 1 equivalent para-toluene sulfonic acid was then added as a 1M stock solution in tetrahydrofuran. The sample was left to mature between room temperature and 50° C. (4 hours at each temperature) for 24 hours with constant shaking before analysis by XRPD.

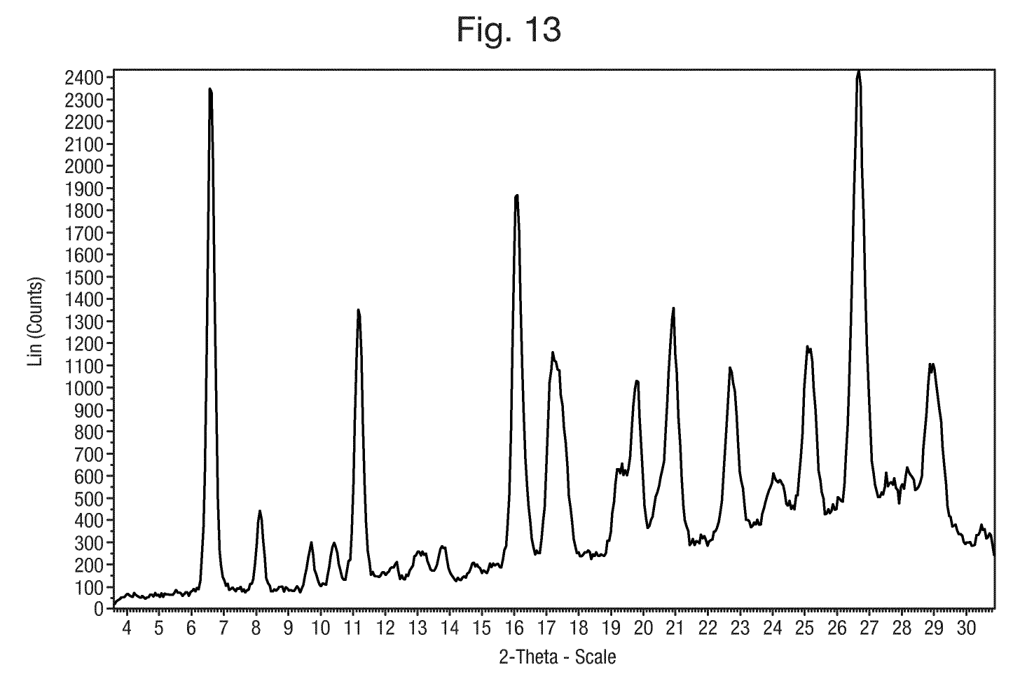

FIG. 13 illustrates the XRPD diffractogram of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate monohydrate. The sample exhibits a good crystallinity.

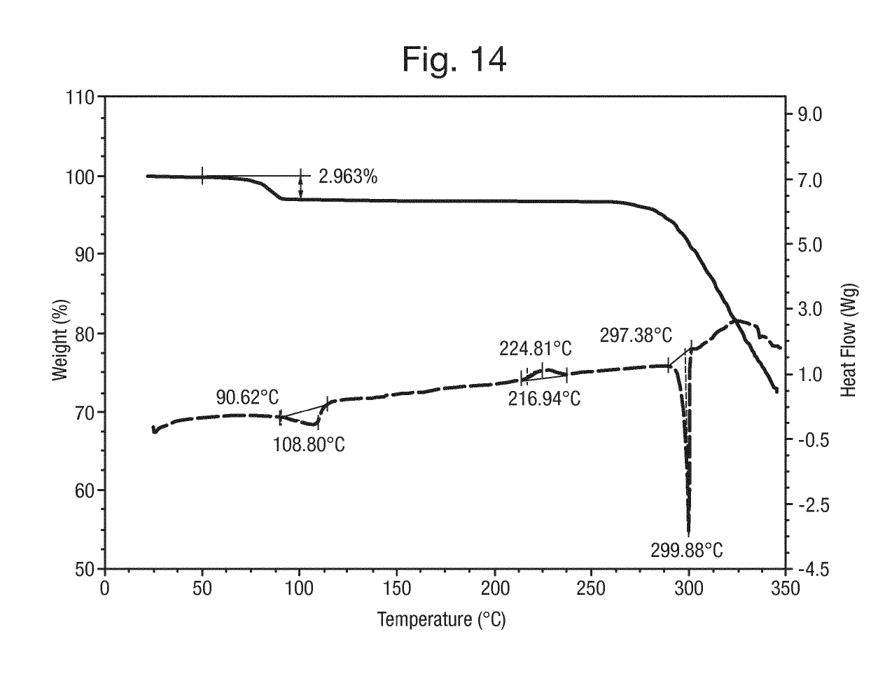

FIG. 14 illustrates the TGA and DSC thermograms of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate monohydrate. The sample showed approx. 2.96% weight loss from 50° C. to 100° C. (equivalent to 1 mol of water). The sample exhibits a small endotherm at 91° C., a small exotherm at 217° C. and a sharp endotherm at 297° C.

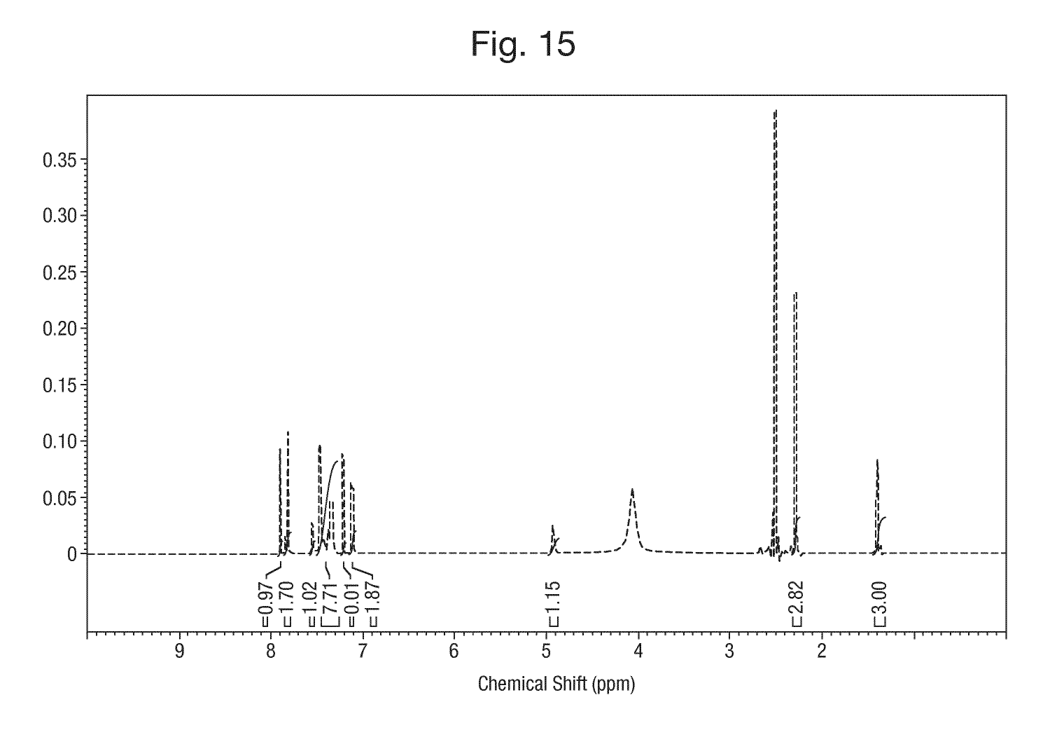

FIG. 15 illustrates the ¹H NMR spectrum of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate monohydrate. The spectra of the sample obtained confirmed the 1:1 stoichiometry of the solid with no residual solvents.

Water-Solubility Test:

The solubility of the of Examples 1-3 in water at room temperature was determined together with the solubility of the corresponding free base. The results are shown in Table 4 below.

| | | Water Solubility |
|---|---|---|
| | | @ 25° C. |
| Ex. | Product | (mg/mL) |
| Ex. 1 | (S)-2-(1-(6-amino-5-cyanopyrimidin-4- | 0.06 |
| | ylamino)ethyl)-4-oxo-3-phenyl-3,4- | |
| | dihydropyrrolo[1,2-f][1,2,4]triazine-5- | |
| | carbonitrile, methanesulfonate | |
| Ex. 2 | (S)-2-(1-(6-amino-5-cyanopyrimidin-4- | 0.07 |
| | ylamino)ethyl)-4-oxo-3-phenyl-3,4- | |
| | dihydropyrrolo[1,2-f][1,2,4]triazine-5- | |
| | carbonitrile, naphthalene-2-sulfonate | |
| Ex. 3 | (S)-2-(1-(6-amino-5-cyanopyrimidin-4- | 0.07 |
| | ylamino)ethyl)-4-oxo-3-phenyl-3,4- | |
| | dihydropyrrolo[1,2-f][1,2,4]triazine-5- | |
| | carbonitrile, para-toluenesulfonate | |
| C1 | (S)-2-(1-(6-amino-5-cyanopyrimidin-4- | 0.001 |
| | ylamino)ethyl)-4-oxo-3-phenyl-3,4- | |
| | dihydropyrrolo[1,2-f][1,2,4]triazine-5- | |
| | carbonitrile | |

As it can be seen from the above results, the salts of the present invention displayed good thermal behaviour, are not hygroscopic, had a relatively high melting point and showed appropriate XRPD pattern before and after GVS determination (no change in form or crystallinity). In addition, the solubility of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile was also improved by preparing the addition salts of the invention, resulting in an improvement of the bioavailability of the free base.

Pharmaceutical Compositions

Pharmaceutical compositions according to the present invention comprise a salt of the invention or pharmaceutically acceptable solvate thereof and a pharmaceutically acceptable carrier.

The salts of the invention are useful in the treatment or prevention of pathological conditions or diseases susceptible to amelioration by inhibition of Phosphoinositide 3-Kinase (PI3K). Such pathological conditions or diseases include but are not limited to respiratory diseases; allergic diseases; inflammatory or autoimmune-mediated; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors.

In particular the pathological conditions or diseases are selected from leukemia, lymphomas and solid tumors, rheumatoid arthritis, multiple sclerosis, amyotrophic lateral sclerosis, Crohn's disease, ulcerative colitis, systemic lupus erythematosis, autoimmune hemolytic anemia, type I diabetes, cutaneous vasculitis, cutaneous lupus erythematosus, dermatomyositis, blistering diseases including but not limited to pemphigus vulgaris, bullous pemphigoid and epidermolysis bullosa, asthma, chronic obstructive pulmonary disease (COPD), cystic fibrosis, bronchiectasis, cough, idiopathic pulmonary fibrosis, sarcoidosis, allergic rhinitis, atopic dermatitis, contact dermatitis, eczema, psoriasis, basal cell carcinoma, squamous cell carcinoma and actinic keratosis.

The pharmaceutical compositions as defined above may further comprise a therapeutically effective amount of one or more other therapeutic agents useful in the treatment or prevention of pathological conditions or diseases susceptible to amelioration by inhibition of Phosphoinositide 3-Kinase (PI3K).

The pharmaceutical compositions of the invention can optionally comprise a therapeutically effective amount one or more additional active substances which are known to be useful in the treatment of respiratory diseases; allergic diseases; inflammatory or autoimmune-mediated; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors; such as such as a) Corticoids and glucocorticoids such as prednisolone, methylprednisolone, dexamethasone, dexamethasone cipecilate, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, butixocort propionate, deprodone propionate, fluticasone propionate, fluticasone furoate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate or hydrocortisone probutate; b) Dyhydrofolate reductase inhibitors, such as methotrexate; c) Dihydroorotate dehydrogenase (DHODH) inhibitors such as leflunomide, teriflunomide, 2-(3′-ethoxy-3-(trifluoromethoxy)biphenyl-4-ylamino)nicotinic acid, 2-(3,5-difluoro-3′-methoxybiphenyl-4-ylamino)nicotinic acid, 2-(3,5-difluoro-2-methylbiphenyl-4-ylamino)nicotinic acid, 5-cyclopropyl-2-(2-(2,6-difluorophenyl)pyrimidin-5-ylamino)benzoic acid, 5-cyclopropyl-2-((2-(2-(trifluoromethyl)phenyl)pyrimidin-5-yl)amino)benzoic acid, 5-methyl-2-((6-(2,3-difluorophenyl)pyridin-3-yl)amino)benzoic acid, and their pharmaceutically acceptable salts; d) Purine analogs, such as Imuran (azathioprine) or Purinethol (6-mercaptopurine or 6-MP); e) Intravenous immunoglobulin (IVIg); f) Antimalarials such as hydroxichloroquine; g) Calcineurin inhibitors such as cyclosporine A or tacrolimus; h) Inosine-monophosphate dehydrogenase (IMPDH) inhibitors, such as mycophenolate mophetyl, ribavirin, mizoribine or mycophenolic acid; i) Immunomodulators such as Glatiramer acetate (Copaxone), Laquinimod or Imiquimod; j) Inhibitors of DNA synthesis and repair, such as Mitoxantrone or Cladribine; k) Fumaric acid esters, such as dimethyl fumarate; l) Interferons comprising Interferon beta 1a, CinnoVex from CinnaGen and Rebif from EMD Serono, and Interferon beta 1b such as Betaferon from Schering and Betaseron from Berlex; m) Interferon alpha such as Sumiferon MP; n) Anti-tumor necrosis factor-alpha (Anti-TNF-alpha) monoclonal antibodies such as Infliximab, Adalimumab or Certolizumab pegol; o) Soluble Tumor necrosis factor-alpha (TNF-alpha) receptors such as Ethanercept; p) Anti-Interleukin 6 Receptor (IL-6R) antibody, such as tocilizumab; q) Anti-Interleukin 12 Receptor (IL-12R)/Interleukin 23 Receptor (IL-23R) antibody, such as ustekinumab; r) Anti-Interleukin 17 Receptor (IL-17R) antibody, such as brodalumab; s) Anti-B-lymphocyte stimulator (BLys) antibodies, such as belimumab; t) Anti-CD20 (lymphocyte protein) antibodies such as Rituximab, Ocrelizumab Ofatumumab or TRU-015; u) Anti-CD52 (lymphocyte protein) antibodies such as alemtuzumab; v) Anti-CD25 (lymphocyte protein) such as daclizumab; w) Anti-CD88 (lymphocyte protein), such as eculizumab or pexilizumab; x) Anti-alpha 4 integrin antibodies, such as natalizumab; y) Anti-Interleukin 5 (IL-5) antibody, such as mepolizumab; z) Anti-Interleukin 5 Receptor (IL-5R) antibody, such as benralizumab; aa) Anti-Interleukin 13 (IL-13) antibody, such as lebrikizumab; bb) Anti-Interleukin 4 Receptor (IL-4R)/Interleukin 13 Receptor (IL-13R) antibody, such as dupilumab; cc) Anti-Interleukin 13 (IL-13)/Interleukin 13 (IL-14) antibody, such as QBX-258; dd) Anti-Interleukin 17 (IL-17) antibody, such as secukinumab; ee) Anti-granulocyte-macrophage colony stimulating factor (GM-CSF) antibodies, such as KBOO3; ff) Anti-Interleukin 1 Receptor (IL-1R) antibody, such as MEDI-8968; gg) Anti-αvβ6 Intregrin, such as STX-100; hh) Anti-Lysyl oxidase-like 2 (LOXL2) antibody, such as Simtuzumab; ii) Anti-connective tissue growth factor (CTGF) antibody, such as FG-3019; jj) Anti-Inmunoglobuline E (IgE) antibody, such as omalizumab; kk) Cytotoxic T lymphocyte antigen 4-Inmunoglobuline (CTLA4-lg) antibody, such as abatacept; ll) Janus kinase (JAK) inhibitors, such as tofacitinib, ruxolitinib, baricitinib, decernotinib, filgotinib, peficitinib, INCB-039110, INCB-047986, ABT-494, INCB-047986 or AC-410; mm) Sphingosine-1 phosphate (S1P) receptor agonists such as fingolimod; nn) Sphingosine-1 phosphate (S1P) liase inhibitors such as LX2931; oo) Spleen tyrosine kinase (Syk) inhibitors, such as R-112; pp) Protein Kinase Inhibitors (PKC) inhibitors, such as NVP-AEB071; q) Nuclear factor-kappaB (NF-kappaB or NFKB) Activation Inhibitors such as Sulfasalazine, Iguratimod or MLN-0415; rr) Epidermal Growth Factor Receptor (EGFR) inhibitors such as erlotinib, Trastuzumab, Herceptin, Avastin, Platins (cisplatin, carboplatin) or Temazolamide; ss) Bruton's tyrosine kinase (Btk) inhibitors, such as ibrutinib; tt) Inhibitors of the Hedgehog signalling pathway, such as vismodegib; uu) Cannabinoid receptor agonists such as Sativex; vv) Chemokine CCR1 antagonists such as MLN-3897 or PS-031291; ww) Chemokine CCR2 antagonists such as INCB-8696; xx) Adenosine A_{2A} agonists, such as ATL-313, ATL-146e, CGS-21680, Regadenoson or UK-432,097; yy) Anti-cholinergic agents such as tiotropium, umeclidinium, glycopyrronium or aclidinium; zz) Beta adrenergic agonists such as salmeterol, formoterol, indacaterol, olodaterol or abediterol; aaa) MABA (molecules with dual activity: beta-adrenergic agonists and muscarinic receptor antagonists); bbb) Histamine 1 (H1) receptor antagonists, such as azelastine or ebastine; ccc) Histamine 4 (H4) receptor antagonists, such as JNJ-38518168; ddd) Cysteinyl leukotriene (CysLT) receptor antagonists, such as montelukast; eee) Mast cell stabilizers, such as nedocromil or chromoglycate; fff) 5-lipoxygenase-activating protein (FLAP) inhibitors, such as MK886 or BAY X 1005; ggg) 5-lipoxygenase (5-LO) inhibitors, such as WY-50295T; hhh) Chemoattractant receptor homologous molecule expressed on TH₂ cells (CRTH2) inhibitors, such as OC-459, AZD-1981, ACT-129968, QAV-680; iii) Vitamin D derivatives like calcipotriol (Daivonex); jjj) Anti-inflammatory agents, such as non-steroidal anti-inflammatory drugs (NSAIDs) or selective cyclooxygenase-2 (COX-2) inhibitors such as aceclofenac, diclofenac, ibuprofen, naproxen, apricoxib, celecoxib, cimicoxib, deracoxib, etoricoxib, lumiracoxib, parecoxib sodium, rofecoxib, selenocoxib-1 or valdecoxib; kkk) Anti-allergic agents; III) Anti-viral agents; mmm) Phosphodiestearase (PDE) III inhibitors; nnn) Phosphosdiesterase (PDE) IV inhibitors such as roflumilast or apremilast; ooo) Dual Phosphodiestearase (PDE) Ill/IV inhibitors; ppp) Phosphodiestearase (PDE) V inhibitors, such as sildenafil; qqq) Xanthine derivatives, such as theophylline or theobromine; rrr) p38 Mitogen-Activated Protein Kinase (p38 MAPK) Inhibitors such as ARRY-797; sss) Mitogen-activated extracellular signal regulated kinase kinase (MEK) inhibitor, such as ARRY-142886 or ARRY-438162; ttt) Antineoplastic agents such as Docetaxel, Estramustine, Anthracyc lines, (doxorubicin (Adriamycin), epirubicin (Ellence), and liposomal doxorubicin (Doxil)), Taxanes (docetaxel (Taxotere), paclitaxel (Taxol), and protein-bound paclitaxel (Abraxane)), Cyclophosphamide (Cytoxan), Capecitabine (Xeloda), 5 fluorouracil (5 FU), Gemcitabine (Gemzar) or Vinorelbine (Navelbine); uuu) Stem cell factor receptor (c-kit) and platelet-derived growth factor (PDGF) receptor inhibitors, such as masitinib; vvv) CXC-chemokine receptor 2 (CXCR2) antagonists, such as AZD5069; www) N-acetylcysteine; xxx) Growth factors receptor inhibitors, such as BIBF1120; yyy) Osmotic regulators such as mannitol and hypertonic saline solution; zzz) Deoxyribonuclease (DNAse), such as pulmozyme; aaaa) Epithelial sodium channel (ENac) inhibitors; bbbb) Potentiators and modulators of CFTR channel; cccc) Neutrophil elastase inhibitors; dddd) Cathepsin C inhibitors. Specific additional active substances that can be combined with the salts of the invention have hereinabove defined.

Combinations

The salts of the invention may also be combined with a therapeutically effective amount of one or more other therapeutic agents useful in the treatment or prevention of pathological conditions or diseases susceptible to amelioration by inhibition of Phosphoinositide 3-Kinase (PI3K).

The combinations of the invention can optionally comprise a therapeutically effective amount one or more additional active substances which are known to be useful in the treatment of respiratory diseases; allergic diseases; inflammatory or autoimmune-mediated; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors; such as a) Corticoids and glucocorticoids such as prednisolone, methylprednisolone, dexamethasone, dexamethasone cipecilate, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, butixocort propionate, deprodone propionate, fluticasone propionate, fluticasone furoate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate or hydrocortisone probutate; b) Dyhydrofolate reductase inhibitors, such as methotrexate; c) Dihydroorotate dehydrogenase (DHODH) inhibitors such as leflunomide, teriflunomide, 2-(3′-ethoxy-3-(trifluoromethoxy)biphenyl-4-ylamino)nicotinic acid, 2-(3,5-difluoro-3′-methoxybiphenyl-4-ylamino)nicotinic acid, 2-(3,5-difluoro-2-methylbiphenyl-4-ylamino)nicotinic acid, 5-cyclopropyl-2-(2-(2,6-difluorophenyl)pyrimidin-5-ylamino)benzoic acid, 5-cyclopropyl-2-((2-(2-(trifluoromethyl)phenyl)pyrimidin-5-yl)amino)benzoic acid, 5-methyl-2-((6-(2,3-difluorophenyl)pyridin-3-yl)amino)benzoic acid, and their pharmaceutically acceptable salts; d) Purine analogs, such as Imuran (azathioprine) or Purinethol (6-mercaptopurine or 6-MP); e) Intravenous immunoglobulin (IVIg); f) Antimalarials such as hydroxichloroquine; g) Calcineurin inhibitors such as cyclosporine A or tacrolimus; h) Inosine-monophosphate dehydrogenase (IMPDH) inhibitors, such as mycophenolate mophetyl, ribavirin, mizoribine or mycophenolic acid; i) Immunomodulators such as Glatiramer acetate (Copaxone), Laquinimod or Imiquimod; j) Inhibitors of DNA synthesis and repair, such as Mitoxantrone or Cladribine; k) Fumaric acid esters, such as dimethyl fumarate; l) Interferons comprising Interferon beta 1a, CinnoVex from CinnaGen and Rebif from EMD Serono, and Interferon beta 1b such as Betaferon from Schering and Betaseron from Berlex; m) Interferon alpha such as Sumiferon MP; n) Anti-tumor necrosis factor-alpha (Anti-TNF-alpha) monoclonal antibodies such as Infliximab, Adalimumab or Certolizumab pegol; o) Soluble Tumor necrosis factor-alpha (TNF-alpha) receptors such as Ethanercept; p) Anti-Interleukin 6 Receptor (IL-6R) antibody, such as tocilizumab; q) Anti-Interleukin 12 Receptor (IL-12R)/Interleukin 23 Receptor (IL-23R) antibody, such as ustekinumab; r) Anti-Interleukin 17 Receptor (IL-17R) antibody, such as brodalumab; s) Anti-B-lymphocyte stimulator (BLys) antibodies, such as belimumab; t) Anti-CD20 (lymphocyte protein) antibodies such as Rituximab, Ocrelizumab Ofatumumab or TRU-015; u) Anti-CD52 (lymphocyte protein) antibodies such as alemtuzumab; v) Anti-CD25 (lymphocyte protein) such as daclizumab; w) Anti-CD88 (lymphocyte protein), such as eculizumab or pexilizumab; x) Anti-alpha 4 integrin antibodies, such as natalizumab; y) Anti-Interleukin 5 (IL-5) antibody, such as mepolizumab; z) Anti-Interleukin 5 Receptor (IL-5R) antibody, such as benralizumab; aa) Anti-Interleukin 13 (IL-13) antibody, such as lebrikizumab; bb) Anti-Interleukin 4 Receptor (IL-4R)/Interleukin 13 Receptor (IL-13R) antibody, such as dupilumab; cc) Anti-Interleukin 13 (IL-13)/Interleukin 13 (IL-14) antibody, such as QBX-258; dd) Anti-Interleukin 17 (IL-17) antibody, such as secukinumab; ee) Anti-granulocyte-macrophage colony stimulating factor (GM-CSF) antibodies, such as KB003; ff) Anti-Interleukin 1 Receptor (IL-1R) antibody, such as MEDI-8968; gg) Anti-αvβ6 Intregrin, such as STX-100; hh) Anti-Lysyl oxidase-like 2 (LOXL2) antibody, such as Simtuzumab; ii) Anti-connective tissue growth factor (CTGF) antibody, such as FG-3019; jj) Anti-Inmunoglobuline E (IgE) antibody, such as omalizumab; kk) Cytotoxic T lymphocyte antigen 4-Inmunoglobuline (CTLA4-lg) antibody, such as abatacept; ll) Janus kinase (JAK) inhibitors, such as tofacitinib, ruxolitinib, baricitinib, decernotinib, filgotinib, peficitinib, INCB-039110, INCB-047986, ABT-494, INCB-047986 or AC-410; mm) Sphingosine-1 phosphate (S1P) receptor agonists such as fingolimod; nn) Sphingosine-1 phosphate (S1P) liase inhibitors such as LX2931; oo) Spleen tyrosine kinase (Syk) inhibitors, such as R-112; pp) Protein Kinase Inhibitors (PKC) inhibitors, such as NVP-AEB071; q) Nuclear factor-kappaB (NF-kappaB or NFKB) Activation Inhibitors such as Sulfasalazine, Iguratimod or MLN-0415; rr) Epidermal Growth Factor Receptor (EGFR) inhibitors such as erlotinib, Trastuzumab, Herceptin, Avastin, Platins (cisplatin, carboplatin) or Temazolamide; ss) Bruton's tyrosine kinase (Btk) inhibitors, such as ibrutinib; tt) Inhibitors of the Hedgehog signalling pathway, such as vismodegib; uu) Cannabinoid receptor agonists such as Sativex; vv) Chemokine CCR1 antagonists such as MLN-3897 or PS-031291; ww) Chemokine CCR2 antagonists such as INCB-8696; xx) Adenosine A_{2A} agonists, such as ATL-313, ATL-146e, CGS-21680, Regadenoson or UK-432,097; yy) Anti-cholinergic agents such as tiotropium, umeclidinium, glycopyrronium or aclidinium; zz) Beta adrenergic agonists such as salmeterol, formoterol, indacaterol, olodaterol or abediterol; aaa) MABA (molecules with dual activity: beta-adrenergic agonists and muscarinic receptor antagonists); bbb) Histamine 1 (H1) receptor antagonists, such as azelastine or ebastine; ccc) Histamine 4 (H4) receptor antagonists, such as JNJ-38518168; ddd) Cysteinyl leukotriene (CysLT) receptor antagonists, such as montelukast; eee) Mast cell stabilizers, such as nedocromil or chromoglycate; fff) 5-lipoxygenase-activating protein (FLAP) inhibitors, such as MK886 or BAY X 1005; ggg) 5-lipoxygenase (5-LO) inhibitors, such as WY-50295T; hhh) Chemoattractant receptor homologous molecule expressed on TH₂ cells (CRTH2) inhibitors, such as OC-459, AZD-1981, ACT-129968, QAV-680; iii) Vitamin D derivatives like calcipotriol (Daivonex); jjj) Anti-inflammatory agents, such as non-steroidal anti-inflammatory drugs (NSAIDs) or selective cyclooxygenase-2 (COX-2) inhibitors such as aceclofenac, diclofenac, ibuprofen, naproxen, apricoxib, celecoxib, cimicoxib, deracoxib, etoricoxib, lumiracoxib, parecoxib sodium, rofecoxib, selenocoxib-1 or valdecoxib; kkk) Anti-allergic agents; ll) Anti-viral agents; mmm) Phosphodiestearase (PDE) III inhibitors; nnn) Phosphosdiesterase (PDE) IV inhibitors such as roflumilast or apremilast; ooo) Dual Phosphodiestearase (PDE) III/IV inhibitors; ppp) Phosphodiestearase (PDE) V inhibitors, such as sildenafil; qqq) Xanthine derivatives, such as theophylline or theobromine; rrr) p38 Mitogen-Activated Protein Kinase (p38 MAPK) Inhibitors such as ARRY-797; sss) Mitogen-activated extracellular signal regulated kinase kinase (MEK) inhibitor, such as ARRY-142886 or ARRY-438162; ttt) Antineoplastic agents such as Docetaxel, Estramustine, Anthracyc lines, (doxorubicin (Adriamycin), epirubicin (Ellence), and liposomal doxorubicin (Doxil)), Taxanes (docetaxel (Taxotere), paclitaxel (Taxol), and protein-bound paclitaxel (Abraxane)), Cyclophosphamide (Cytoxan), Capecitabine (Xeloda), 5 fluorouracil (5 FU), Gemcitabine (Gemzar) or Vinorelbine (Navelbine); uuu) Stem cell factor receptor (c-kit) and platelet-derived growth factor (PDGF) receptor inhibitors, such as masitinib; vvv) CXC-chemokine receptor 2 (CXCR2) antagonists, such as AZD5069; www) N-acetylcysteine; xxx) Growth factors receptor inhibitors, such as BIBF1120; yyy) Osmotic regulators such as mannitol and hypertonic saline solution; zzz) Deoxyribonuclease (DNAse), such as pulmozyme; aaaa) Epithelial sodium channel (ENac) inhibitors; bbbb) Potentiators and modulators of CFTR channel; cccc) Neutrophil elastase inhibitors; dddd) Cathepsin C inhibitors. Specific additional active substances that can be combined with the salts of the invention have hereinabove defined.

The active compounds in the pharmaceutical compositions/combinations of the invention may be administered by any suitable route, depending on the nature of the disorder to be treated, e.g. orally (as syrups, tablets, capsules, lozenges, controlled-release preparations, fast-dissolving preparations, etc); topically (as creams, ointments, lotions, nasal sprays or aerosols, etc); by injection (subcutaneous, intradermic, intramuscular, intravenous, etc.) or by inhalation (as a dry powder, a solution, a dispersion, etc).

The active compounds in the pharmaceutical composition/combination, i.e. the salts of the invention, and the other optional active compounds may be administered together in the same pharmaceutical composition or in different compositions intended for separate, simultaneous, concomitant or sequential administration by the same or a different route.

One execution of the present invention consists of a kit of parts comprising a salt of the invention together with instructions for simultaneous, concurrent, separate or sequential use in combination with another active compound useful in the treatment of respiratory diseases; allergic diseases; inflammatory or autoimmune-mediated; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors; in particular in the treatment of leukemia, lymphomas and solid tumors, rheumatoid arthritis, multiple sclerosis, amyotrophic lateral sclerosis, Crohn's disease, ulcerative colitis, systemic lupus erythematosis, autoimmune hemolytic anemia, type I diabetes, cutaneous vasculitis, cutaneous lupus erythematosus, dermatomyositis, blistering diseases including but not limited to pemphigus vulgaris, bullous pemphigoid and epidermolysis bullosa, asthma, chronic obstructive pulmonary disease (COPD), cystic fibrosis, bronchiectasis, cough, idiopathic pulmonary fibrosis, sarcoidosis, allergic rhinitis, atopic dermatitis, contact dermatitis, eczema, psoriasis, basal cell carcinoma, squamous cell carcinoma and actinic keratosis.

Another execution of the present invention consists of a package comprising a salt of the invention and another active compound useful in the treatment of respiratory diseases; allergic diseases; inflammatory or autoimmune-mediated; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors; in particular in the treatment of leukemia, lymphomas and solid tumors, rheumatoid arthritis, multiple sclerosis, amyotrophic lateral sclerosis, Crohn's disease, ulcerative colitis, systemic lupus erythematosis, autoimmune hemolytic anemia, type I diabetes, cutaneous vasculitis, cutaneous lupus erythematosus, dermatomyositis, blistering diseases including but not limited to pemphigus vulgaris, bullous pemphigoid and epidermolysis bullosa, asthma, chronic obstructive pulmonary disease (COPD), cystic fibrosis, bronchiectasis, cough, idiopathic pulmonary fibrosis, sarcoidosis, allergic rhinitis, atopic dermatitis, contact dermatitis, eczema, psoriasis, basal cell carcinoma, squamous cell carcinoma and actinic keratosis.

The pharmaceutical formulations may conveniently be presented in unit dosage form and may be prepared by any of the methods well known in the art of pharmacy.

Formulations of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A syrup formulation will generally consist of a suspension or solution of the compound or salt in a liquid carrier for example, ethanol, peanut oil, olive oil, glycerine or water with flavouring or colouring agent.

Where the composition is in the form of a tablet, any pharmaceutical carrier routinely used for preparing solid formulations may be used. Examples of such carriers include acacia, lactose, D-glucose (dextrose), sucrose, fructose, galactose, gelatine, starch, calcium carbonate, dibasic calcium phosphate, calcium sulphate, magnesium stearate, magnesium carbonate, isomalt, mannitol, maltitol, stearic acid, sorbitol, talc, xylitol, and mixtures thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Where the composition is in the form of a capsule, any routine encapsulation is suitable, for example using the aforementioned carriers in a hard gelatine capsule. Where the composition is in the form of a soft gelatine capsule any pharmaceutical carrier routinely used for preparing dispersions or suspensions may be considered, for example aqueous gums, celluloses, silicates or oils, and are incorporated in a soft gelatine capsule.

Dry powder compositions for topical delivery to the lung by inhalation may, for example, be presented in capsules and cartridges of for example gelatine or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 2 μg and 150 μg of each therapeutically active ingredient. Alternatively, the active ingredient (s) may be presented without excipients.

Typical compositions for nasal delivery include those mentioned above for inhalation and further include non-pressurized compositions in the form of a solution or suspension in an inert vehicle such as water optionally in combination with conventional excipients such as buffers, anti-microbials, tonicity modifying agents and viscosity modifying agents which may be administered by nasal pump.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

The amount of each active which is required to achieve a therapeutic effect will, of course, vary with the particular active, the route of administration, the subject under treatment, and the particular disorder or disease being treated.

Effective doses are normally in the range of 0.01-2000 mg of active ingredient per day. Daily dosage may be administered in one or more treatments, preferably from 1 to 4 treatments, per day. Preferably, the active ingredients are administered once or twice a day.

When combinations of actives are used, it is contemplated that all active agents would be administered at the same time, or very close in time. Alternatively, one or two actives could be taken in the morning and the other (s) later in the day. Or in another scenario, one or two actives could be taken twice daily and the other (s) once daily, either at the same time as one of the twice-a-day dosing occurred, or separately. Preferably at least two, and more preferably all, of the actives would be taken together at the same time. Preferably, at least two, and more preferably all actives would be administered as an admixture.

The following preparations forms are cited as composition (formulation) examples are given in order to provide a person skilled in the art with a sufficiently clear and complete explanation of the present invention, but should not be considered as limiting of the essential aspects of its subject, as set out in the preceding portions of this description.

### Composition Example 1

50,000 capsules, each containing 100 mg (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile, methanesulfonate (active ingredient), were prepared according to the following formulation:

| | |
|---|---|
| Active ingredient | 5 Kg |
| Lactose monohydrate | 10 Kg |
| Colloidal silicon dioxide | 0.1 Kg |
| Corn starch | 1 Kg |
| Magnesium stearate | 0.2 Kg |

Procedure

The above ingredients were sieved through a 60 mesh sieve, and were loaded into a suitable mixer and filled into 50,000 gelatine capsules.

### Composition Example 2

50,000 tablets, each containing 50 mg of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile, methanesulfonate (active ingredient), were prepared from the following formulation:

| | |
|---|---|
| Active ingredient | 2.5 Kg |
| Microcrystalline cellulose | 1.95 Kg |
| Spray dried lactose | 9.95 Kg |
| Carboxymethyl starch | 0.4 Kg |
| Sodium stearyl fumarate | 0.1 Kg |
| Colloidal silicon dioxide | 0.1 Kg |

Procedure All the powders were passed through a screen with an aperture of 0.6 mm, then mixed in a suitable mixer for 20 minutes and compressed into 300 mg tablets using 9 mm disc and flat bevelled punches. The disintegration time of the tablets was about 3 minutes.

### Composition Example 3

| Ingredient | Amount |
|---|---|
| S)-2-(1-(6-amino-5-cyanopyrimidin-4- | 1% |
| ylamino)ethyl)-4-oxo-3-phenyl-3,4- | |
| dihydropyrrolo[1,2-f][1,2,4]triazine-5- | |
| carbonitrile, methanesulfonate | |
| Cetyl alcohol | 3% |
| Stearyl alcohol | 4% |
| Glyceryl monostearate | 4% |
| Sorbitan monostearate | 0.8% |
| Sorbitan monostearate POE | 0.8% |
| Liquid Vaseline | 0.8% |
| Glycerine | 15% |
| Preservative | 0.2% |
| Purified water | add to 100% |

An oil-in-water emulsion cream was prepared with the ingredients listed above, using conventional methods.

Modifications, which do not affect, alter, change or modify the essential aspects of the compounds, combinations or pharmaceutical compositions described, are included within the scope of the present invention.

1. A pharmaceutically acceptable crystalline addition salt of (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile with sulfonic acid derivatives chosen from methanesulfonic acid, naphthalene-2-sulfonic acid and para-toluenesulfonic acid, or a pharmaceutically acceptable solvate thereof.

2. The salt according to claim 1, chosen from (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile methanesulfonate, or a pharmaceutically acceptable solvate thereof.

3. The salt according to claim 1, chosen from (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate, or a pharmaceutically acceptable solvate thereof.

4. The salt according to claim 1, chosen from (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate, or a pharmaceutically acceptable solvate thereof.

5. A pharmaceutical composition comprising a therapeutically effective amount of the salt according to claim 1 and a pharmaceutically acceptable carrier.

6. The pharmaceutical composition according to claim 5 further comprising a therapeutically effective amount of at least one additional therapeutic agent.

7. The pharmaceutical composition according to claim 6, wherein the at least one additional therapeutic agent is chosen from:
a) Corticoids and glucocorticoids,
b) Dyhydrofolate reductase inhibitors,
c) Dihydroorotate dehydrogenase (DHODH) inhibitors,
d) Purine analogs,
e) Intravenous immunoglobulin (IVIg),
f) Antimalarials such as hydroxichloroquine,
g) Calcineurin inhibitors,
h) Inosine-monophosphate dehydrogenase (IMPDH) inhibitors,
i) Immunomodulators,
j) Inhibitors of DNA synthesis and repair,
k) Fumaric acid esters,
l) Interferons comprising Interferon beta 1a, and Interferon beta 1b,
m) Interferon alpha,
n) Anti-tumor necrosis factor-alpha (Anti-TNF-alpha) monoclonal antibodies,
o) Soluble Tumor necrosis factor-alpha (TNF-alpha) receptors,
p) Anti-Interleukin 6 Receptor (IL-6R) antibody,
q) Anti-Interleukin 12 Receptor (IL-12R)/Interleukin 23 Receptor (IL-23R) antibody,
r) Anti-Interleukin 17 Receptor (IL-17R) antibody,
s) Anti-B-lymphocyte stimulator (BLys) antibodies,
t) Anti-CD20 (lymphocyte protein) antibodies,
u) Anti-CD52 (lymphocyte protein) antibodies,
v) Anti-CD25 (lymphocyte protein),
w) Anti-CD88 (lymphocyte protein),
x) Anti-alpha 4 integrin antibodies,
y) Anti-Interleukin 5 (IL-5) antibody,
z) Anti-Interleukin 5 Receptor (IL-5R) antibody,
aa) Anti-Interleukin 13 (IL-13) antibody,
bb) Anti-Interleukin 4 Receptor (IL-4R)/Interleukin 13 Receptor (IL-13R) antibody,
cc) Anti-Interleukin 13 (IL-13)/Interleukin 13 (IL-14) antibody,
dd) Anti-Interleukin 17 (IL-17) antibody,
ee) Anti-granulocyte-macrophage colony stimulating factor (GM-CSF) antibodies,
ff) Anti-Interleukin 1 Receptor (IL-1R) antibody,
gg) Anti-αvβ6 Intregrin,
hh) Anti-Lysyl oxidase-like 2 (LOXL2) antibody,
ii) Anti-connective tissue growth factor (CTGF) antibody,
jj) Anti-Inmunoglobuline E (IgE) antibody,
kk) Cytotoxic T lymphocyte antigen 4-Inmunoglobuline (CTLA4-lg) antibody,
ll) Janus kinase (JAK) inhibitors,
mm) Sphingosine-1 phosphate (S1P) receptor agonists,
nn) Sphingosine-1 phosphate (S1P) liase inhibitors,
oo) Spleen tyrosine kinase (Syk) inhibitors,
pp) Protein Kinase Inhibitors (PKC) inhibitors,
qq) Nuclear factor-kappaB (NF-kappaB or NFKB) Activation Inhibitors,
rr) Epidermal Growth Factor Receptor (EGFR) inhibitors,
ss) Bruton's tyrosine kinase (Btk) inhibitors,
tt) Inhibitors of the Hedgehog signalling pathway,
uu) Cannabinoid receptor agonists,
vv) Chemokine CCR1 antagonists,
ww) Chemokine CCR2 antagonists,
xx) Adenosine A_{2A} agonists,
yy) Anti-cholinergic agents,
zz) Beta adrenergic agonists,
aaa) MABA (molecules with dual activity: beta-adrenergic agonists and muscarinic receptor antagonists),
bbb) Histamine 1 (H1) receptor antagonists,
ccc) Histamine 4 (H4) receptor antagonists,
ddd) Cysteinyl leukotriene (CysLT) receptor antagonists,
eee) Mast cell stabilizers,
fff) 5-lipoxygenase-activating protein (FLAP) inhibitors,
ggg) 5-lipoxygenase (5-LO) inhibitors,
hhh) Chemoattractant receptor homologous molecule expressed on TH₂ cells (CRTH2) inhibitors,
iii) Vitamin D derivatives,
jjj) Anti-inflammatory agents, or selective cyclooxygenase-2 (COX-2) inhibitors,
kkk) Anti-allergic agents,
lll) Anti-viral agents,
mmm) Phosphosdiesterase (PDE) III inhibitors,
nnn) Phosphosdiesterase (PDE) IV inhibitors,
ooo) Dual Phosphosdiesterase (PDE) III/IV inhibitors,
ppp) Phosphosdiesterase (PDE) V inhibitors,
qqq) Xanthine derivatives,
rrr) p38 Mitogen-Activated Protein Kinase (p38 MAPK) Inhibitors,
sss) Mitogen-activated extracellular signal regulated kinase kinase (MEK) inhibitor,
ttt) Antineoplastic agents,
uuu) Stem cell factor receptor (c-kit) and platelet-derived growth factor (PDGF) receptor inhibitors,
vvv) CXC-chemokine receptor 2 (CXCR2) antagonists,
www) N-acetylcysteine,
xxx) Growth factors receptor inhibitors,
yyy) Osmotic regulators,
zzz) Deoxyribonuclease (DNAse),
aaaa) Epithelial sodium channel (ENac) inhibitors;
bbbb) Potentiators and modulators of CFTR channel,
cccc) Neutrophil elastase inhibitors, and
dddd) Cathepsin C inhibitors.

8. A combination comprising a salt according to claim 1 and at least one additional therapeutic agent chosen from:
a) Corticoids and glucocorticoids,
b) Dyhydrofolate reductase inhibitors,
c) Dihydroorotate dehydrogenase (DHODH) inhibitors,
d) Purine analogs,
e) Intravenous immunoglobulin (IVIg),
f) Antimalarials such as hydroxichloroquine,
g) Calcineurin inhibitors,
h) Inosine-monophosphate dehydrogenase (IMPDH) inhibitors,
i) Immunomodulators,
j) Inhibitors of DNA synthesis and repair,
k) Fumaric acid esters,
l) Interferons comprising Interferon beta 1a, and Interferon beta 1b,
m) Interferon alpha,
n) Anti-tumor necrosis factor-alpha (Anti-TNF-alpha) monoclonal antibodies,
o Soluble Tumor necrosis factor-alpha (TNF-alpha) receptors,
p) Anti-Interleukin 6 Receptor (IL-6R) antibody,
g) Anti-Interleukin 12 Receptor (IL-12R)/Interleukin 23 Receptor (IL-23R) antibody,
r) Anti-Interleukin 17 Receptor (IL-17R) antibody,
s) Anti-B-lymphocyte stimulator (BLys) antibodies,
t) Anti-CD20 (lymphocyte protein) antibodies,
u) Anti-CD52 (lymphocyte protein) antibodies,
v) Anti-CD25 (lymphocyte protein),
w) Anti-CD88 (lymphocyte protein),
x) Anti-alpha 4 integrin antibodies,
y) Anti-Interleukin 5 (IL-5) antibody,
z) Anti-Interleukin 5 Receptor (IL-5R) antibody,
aa) Anti-Interleukin 13 (IL-13) antibody,
bb) Anti-Interleukin 4 Receptor (IL-4R)/Interleukin 13 Receptor (IL-13R) antibody,
cc) Anti-Interleukin 13 (IL-13)/Interleukin 13 (IL-14) antibody,
dd) Anti-Interleukin 17 (IL-17) antibody,
ee) Anti-granulocyte-macrophage colony stimulating factor (GM-CSF) antibodies,
ff) Anti-Interleukin 1 Receptor (IL-1R) antibody,
gg) Anti-αvβ6 Intregrin,
hh) Anti-Lysyl oxidase-like 2 (LOXL2) antibody,
ii) Anti-connective tissue growth factor (CTGF) antibody,
jj) Anti-Inmunoglobuline E (IqE) antibody,
kk) Cytotoxic T lymphocyte antigen 4-Inmunoglobuline (CTLA4-Iq) antibody,
ll) Janus kinase (JAK) inhibitors,
mm) Sphingosine-1 phosphate (S1P) receptor agonists,
nn) Sphingosine-1 phosphate (S1P) liase inhibitors,
oo) Spleen tyrosine kinase (Syk) inhibitors,
pp) Protein Kinase Inhibitors (PKC) inhibitors,
qq) Nuclear factor-kappaB (NF-kappaB or NFKB) Activation Inhibitors,
rr) Epidermal Growth Factor Receptor (EGFR) inhibitors,
ss) Bruton's tyrosine kinase (Btk) inhibitors,
tt) Inhibitors of the Hedgehog signaling pathway,
uu) Cannabinoid receptor agonists,
vv) Chemokine CCR1 antagonists,
ww) Chemokine CCR2 antagonists,
xx) Adenosine A_{2A} agonists,
yy) Anti-cholinergic agents,
zz) Beta adrenergic agonists,
aaa) MABA (molecules with dual activity: beta-adrenergic agonists and muscarinic receptor antagonists),
bbb) Histamine 1 (H1) receptor antagonists,
ccc) Histamine 4 (H4) receptor antagonists,
ddd) Cysteinyl leukotriene (CysLT) receptor antagonists,
eee) Mast cell stabilizers,
fff) 5-lipoxygenase-activating protein (FLAP) inhibitors,
ggg) 5-lipoxygenase (5-LO) inhibitors,
hhh) Chemoattractant receptor homologous molecule expressed on TH₂ cells (CRTH2) inhibitors,
iii) Vitamin D derivatives,
jjj) Anti-inflammatory agents, or selective cyclooxygenase-2 (COX-2) inhibitors,
kkk) Anti-allergic agents,
lll) Anti-viral agents,
mmm) Phosphodiesterase (PDE) III inhibitors,
nnn) Phosphosdiesterase (PDE) IV inhibitors,
ooo) Dual Phosphodiesterase (PDE) III/IV inhibitors,
ppp) Phosphodiesterase (PDE) V inhibitors,
ggg) Xanthine derivatives,
rrr) p38 Mitogen-Activated Protein Kinase (p38 MAPK) Inhibitors,
sss) Mitogen-activated extracellular signal regulated kinase kinase (MEK) inhibitor,
ttt) Antineoplastic agents,
uuu) Stem cell factor receptor (c-kit) and platelet-derived growth factor (PDGF) receptor inhibitors,
vvv) CXC-chemokine receptor 2 (CXCR2) antagonists,
www) N-acetylcysteine,
xxx) Growth factors receptor inhibitors,
yyy) Osmotic regulators,
zzz) Deoxyribonuclease (DNAse),
aaaa) Epithelial sodium channel (ENac) inhibitors;
bbbb) Potentiators and modulators of CFTR channel,
cccc) Neutrophil elastase inhibitors, and
dddd) Cathepsin C inhibitors.

9. A method for treating a subject afflicted with a pathological condition or disease susceptible to amelioration by inhibition of Phosphoinositide 3-Kinase (PI3K), comprising administering to the subject a therapeutically effective amount of the salt according to claim 1.

10. The method according to claim 9, wherein the pathological condition or disease is chosen from respiratory diseases; allergic diseases; inflammatory or autoimmune-mediated diseases; function disorders and neurological disorders; cardiovascular diseases; viral infection; metabolism/endocrine function disorders; neurological disorders and pain; bone marrow and organ transplant rejection; myelo-dysplastic syndrome; myeloproliferative disorders (MPDs); cancer and hematologic malignancies, leukemia, lymphomas and solid tumors.

11. The method according to claim 9, wherein the pathological condition or disease is chosen from leukemia, lymphomas and solid tumors, rheumatoid arthritis, multiple sclerosis, amyotrophic lateral sclerosis, Crohn's disease, ulcerative colitis, systemic lupus erythematosis, autoimmune hemolytic anemia, type I diabetes, cutaneous vasculitis, cutaneous lupus erythematosus, dermatomyositis, blistering diseases including but not limited to pemphigus vulgaris, bullous pemphigoid and epidermolysis bullosa, asthma, chronic obstructive pulmonary disease (COPD), cystic fibrosis, bronchiectasis, cough, idiopathic pulmonary fibrosis, sarcoidosis, allergic rhinitis, atopic dermatitis, contact dermatitis, eczema, psoriasis, basal cell carcinoma, squamous cell carcinoma and actinic keratosis.

12. (canceled)

13. A method for treating a subject afflicted with a pathological condition or disease susceptible to amelioration by inhibition of PI3K, comprising administering to the subject the a pharmaceutical composition according to claim 5.
